(19) United States
(12) Patent Application Publication
(10) Pub. No.: US 2025/0090621 A1
(43) Pub. Date: Mar. 20, 2025

(54) METHODS AND COMPOSITIONS FOR THE TREATMENT OF AGE-RELATED MACULAR DEGENERATION

(71) Applicant: Stealth BioTherapeutics Inc., Needham, MA (US)

(72) Inventors: Anthony Abbruscato, Needham, MA (US); Michael Dimatteo, Needham, MA (US)

(73) Assignee: Stealth BioTherapeutics Inc., Needham, MA (US)

(21) Appl. No.: 18/963,148

(22) Filed: Nov. 27, 2024

Related U.S. Application Data
(63) Continuation of application No. PCT/US2024/032574, filed on Jun. 5, 2024.
(60) Provisional application No. 63/471,351, filed on Jun. 6, 2023.
(60) Provisional application No. 63/615,153, filed on Dec. 27, 2023.

(51) Int. Cl. A61K 38/07; A61B 3/10; A61K 45/06; A61P 27/02

(52) U.S. Cl. CPC A61K 38/07; A61B 3/102; A61K 45/06; A61P 27/02

(57) ABSTRACT

The present technology provides methods and compositions for treating, preventing and/or delaying (slowing) the progression of age-related macular degeneration (AMD). In particular, the present technology relates to the use of elamipretide in effective amounts to treat, prevent and/or delay (slow) the progression of photoreceptor loss, AMD, GA secondary to AMD, non-exudative (dry) age-related macular degeneration, intermediate age-related macular degeneration, age-related macular degeneration with photoreceptor loss or non-exudative (dry) age-related macular degeneration with photoreceptor loss in mammalian subjects.

### CROSS-REFERENCE TO RELATED APPLICATIONS

This application claims the benefit of and priority to U.S. Provisional Patent Application No. 63/471,351, filed Jun. 6, 2023, and U.S. Provisional Patent Application No. 63/615,153, filed Dec. 27, 2023, the contents of which are incorporated herein by reference in their entireties.

### TECHNICAL FIELD

The present technology relates generally to methods and compositions for treating, preventing, or delaying (slowing) the progression of (wet or dry) age-related macular degeneration (AMD). In particular, the present technology relates to the use of elamipretide in effective amounts to treat, prevent and/or delay (slow) the progression of photoreceptor loss, AMD, geographic atrophy (GA) secondary to AMD, non-exudative (a.k.a., dry) AMD, intermediate AMD, AMD with photoreceptor loss or non-exudative (a.k.a., dry) AMD with photoreceptor loss in mammalian subjects.

### INTRODUCTION

The following description is provided to assist the understanding of the reader. None of the information provided or references cited is admitted to be prior art to the compositions and methods disclosed herein.

Age-related macular degeneration (AMD) affects ˜11 million Americans and is the leading cause of irreversible blindness in people aged ≥50 years. (Pennington 2016) AMD prevalence (United States) is estimated to increase to over 20 million patients by 2050. (Rein 2009) AMD preferentially affects the macular (central) region of the retina, and is characterized as early, intermediate, or late stages based on number, location, and size of drusen with hyper- or hypopigmentary changes and the presence or absence of geographic atrophy (GA) or macular neovascularization (MNV) or choroidal neovascularization (CNV). Late stages of non-exudative (or dry) AMD are characterized by GA, which is an advanced form of AMD and leads to progressive and irreversible loss of visual function (Fleckenstein 2018). GA has a major negative impact on vision-related quality of life (QoL) (Chakravarthy 2018) and accounts for 20% and 25% of legal blindness in the US and UK, respectively (Holz 2014; Boyer 2017). Geographic atrophy is defined by the presence of sharply demarcated atrophic lesions of the outer retina, resulting from loss of photoreceptors, retinal pigment epithelium (RPE), and underlying choriocapillaris (Fleckenstein 2018). There is a need for better methods, compounds and compositions for the treatment, prevention and/or delaying (slowing) the progression of AMD.

### SUMMARY

In one aspect, the present disclosure provides a method for treating, preventing or delaying (slowing) the progression of (wet or dry) age-related macular degeneration (AMD) in a mammalian subject in need thereof, comprising the steps of: a) selecting subjects diagnosed as having or suspected of having AMD; b) examining one or both eyes of the subject using optical coherence tomography (OCT) to produce a first OCT scan for each eye examined; c) analyzing each OCT scan to determine: (i) the area percent of partial EZ attenuation (pEZa); (ii) the area percent of total EZ attenuation (tEZa); and/or (iii) the center 1-mm ellipsoid zone-retinal pigment epithelium (EZ-RPE) thickness; and d) administering an effective amount of elamipretide to subjects presenting with AMD in at least one eye; (i) an area percent of partial EZ attenuation (pEZa) greater than zero and less than 100 percent; (ii) an area percent of total EZ attenuation (tEZa) is greater than zero and less than 100 percent; and/or (iii) a center 1-mm EZ-RPE thickness is greater than 20 μm. In some embodiments, the method is directed towards treating, preventing or delaying (slowing) the progression of wet (i.e., exudative) age-related macular degeneration (AMD). In some embodiments, the method is directed towards treating, preventing or delaying (slowing) the progression of dry (i.e., non-exudative) age-related macular degeneration (AMD).

In some embodiments, the elamipretide is administered to the subject on a daily, weekly, or monthly basis.

In some embodiments, the area percent of tEZa is greater than zero and less than 50 percent. In some embodiments, the area percent of tEZa is greater than zero and less than 30 percent. In some embodiments, the area percent of tEZa is greater than zero and less than 20 percent. In some embodiments, the subject has high-risk drusen (HRD).

In some embodiments, analyzing the first OCT scan comprises evaluating higher-order OCT features using automated machine-learning augmented multilayer retinal segmentation optionally with expert manual reader verification.

In some embodiments, the subject is also evaluated using a visual acuity test and at baseline has a best corrected visual acuity (BCVA) in at least one eye of at least 55 letters and a low luminance best corrected visual acuity (LL-BCVA) deficit of at least 5 letters.

In some embodiments, administration of elamipretide leads to at least a 2-line gain (i.e., at least 10 letters) in LL BCVA from baseline in the subject after at least one year of elamipretide administration/treatment. In some embodiments, administration of elamipretide leads to at least a 3-line gain (i.e., at least 15 letters) in LL BCVA from baseline in the subject after at least one year of elamipretide administration.

In some embodiments, the method further comprises examining one or both eyes of the subject using OCT to produce a second OCT scan for each eye examined.

In some embodiments, analyzing each second OCT scan comprises evaluating higher-order OCT features using automated machine-learning augmented multilayer retinal segmentation optionally with expert manual reader verification.

In some embodiments, the subject is a human.

In some embodiments, the elamipretide is administered intraocularly, iontophoretically, orally, topically, systemically, intravenously, subcutaneously, or intramuscularly.

In some embodiments, the method further comprises separately, sequentially, or simultaneously administering a second active agent.

In some embodiments, the second active agent comprises an AREDS or AREDS 2 vitamin formula. In some embodiments, the second active agent is selected from the group consisting of: an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, or a carotenoid. In some embodiments, the second active agent is mometasone furoate, tacrolimus, quercetin, or diphenhydramine.

In some embodiments, the second active agent is flavonoid, a coumarin, a phenol or a terpenoid. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

In some embodiments, about 5 mg to about 80 mg of elamipretide, or a pharmaceutically acceptable salt thereof, is subcutaneously administered to the injection site of the subject.

In some embodiments, about 10 mg to about 60 mg of elamipretide, or a pharmaceutically acceptable salt thereof, is subcutaneously administered to the injection site of the subject.

In some embodiments, about 20 mg to about 60 mg of elamipretide, or a pharmaceutically acceptable salt thereof, is subcutaneously administered to the injection site of the subject.

In some embodiments, about 20 mg of elamipretide, or a pharmaceutically acceptable salt thereof, is subcutaneously administered to the injection site of the subject.

In some embodiments, about 40 mg of elamipretide, or a pharmaceutically acceptable salt thereof, is subcutaneously administered to the injection site of the subject.

In some embodiments, 40 mg of elamipretide, or a pharmaceutically acceptable salt thereof, is subcutaneously administered to the injection site of the subject.

In some embodiments, about 60 mg of elamipretide, or a pharmaceutically acceptable salt thereof, is subcutaneously administered to the injection site of the subject.

In some embodiments, 60 mg of elamipretide, or a pharmaceutically acceptable salt thereof, is subcutaneously administered to the injection site of the subject.

In some embodiments, the subject presents with an area percent of partial EZ attenuation (pEZa) is greater than 5 and less than 80 percent.

In some embodiments, the subject presents with a center 1-mm EZ-RPE thickness that is greater than 20 μm.

In some embodiments, the aforementioned method further provides for treating, preventing or delaying (slowing) the progression of geographic atrophy (GA) secondary to age-related macular degeneration (AMD). In some embodiments, the aforementioned method further provides for treating, preventing or delaying (slowing) the progression of non-exudative (dry) age-related macular degeneration. In some embodiments, the aforementioned method provides for treating, preventing or delaying (slowing) the progression of intermediate age-related macular degeneration. In some embodiments, the aforementioned method further provides for treating, preventing or delaying (slowing) the progression of age-related macular degeneration with photoreceptor loss or non-exudative (dry) age-related macular degeneration with photoreceptor loss. In some embodiments, the aforementioned method further provides for treating, preventing or delaying (slowing) the progression of age-related macular degeneration by slowing the rate of photoreceptor loss.

In one aspect, the present disclosure provides a method comprising: a) examining one or both eyes of a mammalian subject using optical coherence tomography (OCT) to produce a 1^{st} OCT scan for each eye examined; b) reexamining one or both eyes of the subject using optical coherence tomography (OCT) to produce a 2^{nd} OCT scan for one or both of the subject's eyes on a day that is after performing the examination according to step (a); c) optionally repeating step (b) n-times, each n^{th} reexamination occurring on a day that is after the n^{th}−1 examination, that produced an n^{th}−1 OCT scan, to thereby produce a n^{th} OCT scan for one or both eyes, where n is a whole number from 3 to 100; and d) examining the OCT scans to determine: (i) a rate of photoreceptor loss; (ii) a percent of photoreceptor loss; and/or (iii) a mean change in the macular area of photoreceptor loss; in each eye examined for the subject that has occurred from: a) the 1^{st} OCT scan to the 2^{nd} OCT scan or to any one or more of the n^{th} OCT scans; and/or b) between two later obtained OCT scans for a particular eye examined.

In some embodiments, the method further comprises prescribing the administration of elamipretide for the subject to thereby treat, prevent or delay (slow) progression of the subject's photoreceptor loss in one or both eyes. In some embodiments, the method further comprises identifying the subject as a candidate for the administration of elamipretide to thereby treat, prevent or delay (slow) progression of the subject's photoreceptor loss in one or both eyes. In some embodiments, the method further comprises administering elamipretide to the subject to thereby treat, prevent or delay (slow) progression of the subject's photoreceptor loss in one or both eyes. In some embodiments, elamipretide is administered subcutaneously to the subject once daily at 40 mg/dose.

In some embodiments, the 2^{nd} OCT scan is produced between 1 month and 3 months, or 3 months and 6 months, or 6 months and one year, after the 1^{st} OCT scan is produced. In some embodiments, each n^{th} OCT scan is produced between 1 month and 3 months, or 3 months and 6 months, or 6 months and one year, after the n^{th}−1 OCT scan is produced.

In some embodiments, delaying (slowing) progression of the subject's photoreceptor loss in one or both eyes corresponds with delaying (slowing) progression of (wet or dry) age-related macular degeneration (AMD) in one or both of the subject's eyes. In some embodiments, delaying (slowing) progression of the subject's photoreceptor loss in one or both eyes correspond with delaying (slowing) progression of geographic atrophy (GA) secondary to age-related macular degeneration (AMD) in one or both of the subject's eyes.

In some embodiments, the method further provides for treating, preventing or delaying (slowing) the progression of non-exudative (dry) age-related macular degeneration in one or both of the subject's eyes. In some embodiments, the method further provides for treating, preventing or delaying (slowing) the progression of intermediate age-related macular degeneration in one or both of the subject's eyes. In some embodiments, the method further provides for treating, preventing or delaying (slowing) the progression of age-related macular degeneration with photoreceptor loss or non-exudative (dry) age-related macular degeneration with photoreceptor loss in one or both of the subject's eyes. In some embodiments, the method further provides for treating, preventing or delaying (slowing) the progression of age-related macular degeneration by slowing the rate of photoreceptor loss in one or both of the subject's eyes.

In some embodiments, the method further comprises separately, sequentially, or simultaneously administering a second active agent.

In some embodiments, the second active agent comprises an AREDS or AREDS 2 vitamin formula. In some embodiments, the second active agent is selected from the group consisting of: an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, or a carotenoid. In some embodiments, the second active agent is mometasone furoate, tacrolimus, quercetin, or diphenhydramine.

In some embodiments, the second active agent is flavonoid, a coumarin, a phenol or a terpenoid. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

In one aspect, the present disclosure provides a method comprising: a) examining one or both eyes of a mammalian subject using a visual acuity test to determine the subject's 1^{st} low luminance best corrected visual acuity (1^{st} LL BCVA), collectively and/or on an eye-by-eye basis; b) reexamining one or both eyes of the subject using a visual acuity test, to thereby determine the subject's 2^{nd} low luminance best corrected visual acuity (2^{nd} LL BCVA), collectively and/or on an eye-by-eye basis, on a day that is after performing the examination according to step (a); c) optionally repeating step (b) n-times, to thereby determine the subject's n^{th} low luminance best corrected visual acuity (n^{th} LL BCVA), collectively and/or on an eye-by-eye basis, each n^{th} reexamination occurring on a day that is after the n^{th}−1 examination, where n is a whole number from 3 to 100; and d) determining whether the subject has lost or gained one or more letters and/or one or more lines in LL BCVA, collectively or on an eye-by-eye basis: from the 1^{st} LL BCVA determination to the 2^{nd} LL BCVA determination or any one or more of the n^{th} LL BCVA determinations; and/or b) between two later LL BCVA determinations.

In some embodiments, the method further comprises prescribing the administration of elamipretide for the subject to thereby treat, prevent or delay (slow) progression of the subject's loss in LL BCVA, collectively or on an eye-by-eye basis. In some embodiments, the method further comprises identifying the subject as a candidate for the administration of elamipretide to thereby treat, prevent or delay (slow) progression of the subject's loss in LL BCVA, collectively or on an eye-by-eye basis. In some embodiments, the method further comprises administering elamipretide to the subject to thereby treat, prevent or delay (slow) progression of the subject's loss in LL BCVA, collectively or on an eye-by-eye basis. In some embodiments, elamipretide is administered subcutaneously to the subject once daily at 40 mg/dose.

In some embodiments of the method, the 2^{nd} LL BCVA determination is performed between 1 month and 3 months, or 3 months and 6 months, or 6 months and one year, after the 1S^{t} LL BCVA determination is performed. In some embodiments, each n^{th} LL BCVA determination is performed between 1 month and 3 months, or 3 months and 6 months, or 6 months and one year, after the n^{th}-LL BCVA determination is performed. In some embodiments, the subject's LL BCVA is determined on an annual basis.

In some embodiments, delaying (slowing) progression of the subject's loss in LL BCVA in one or both eyes correspond with delaying (slowing) progression of age-related macular degeneration (AMD) in one or both of the subject's eyes. In some embodiments, delaying (slowing) progression of the subject's loss in LL BCVA in one or both eyes correspond with delaying (slowing) progression of geographic atrophy (GA) secondary to age-related macular degeneration (AMD) in one or both of the subject's eyes.

In some embodiments, the method further provides for treating, preventing or delaying (slowing) the progression of non-exudative (dry) age-related macular degeneration in one or both of the subject's eyes. In some embodiments, the method further provides for treating, preventing or delaying (slowing) the progression of intermediate age-related macular degeneration in one or both of the subject's eyes. In some embodiments, the method further provides for treating, preventing or delaying (slowing) the progression of age-related macular degeneration with photoreceptor loss or non-exudative (dry) age-related macular degeneration with photoreceptor loss in one or both of the subject's eyes. In some embodiments, the method further provides for treating, preventing or delaying (slowing) the progression of age-related macular degeneration by slowing the rate of photoreceptor loss in one or both of the subject's eyes.

In some embodiments, the method further comprises separately, sequentially, or simultaneously administering a second active agent.

In some embodiments, the second active agent comprises an AREDS or AREDS 2 vitamin formula. In some embodiments, the second active agent is selected from the group consisting of: an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, or a carotenoid. In some embodiments, the second active agent is mometasone furoate, tacrolimus, quercetin, or diphenhydramine.

In some embodiments, the second active agent is flavonoid, a coumarin, a phenol or a terpenoid. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

In one aspect, the present disclosure provides a method for improving the low luminance best corrected visual acuity (LL BCVA) by 2 or more lines, collectively or on an eye-by-eye basis, in a mammalian subject having, or suspected of having, age-related macular degeneration (AMD), comprising administering elamipretide to the subject for a period of at least 12 weeks. In some embodiments, the elamipretide is administered subcutaneously. In some embodiments, the elamipretide is administered once daily at 40 mg/dose. In some embodiments, the subject's low luminance best corrected visual acuity (LL BCVA) is increased by 3 or more lines, by 4 or more lines or by 5 or more lines in one or both of the subject's eyes. In some embodiments, the subject has been diagnosed as having geographic atrophy (GA) secondary to age-related macular degeneration (AMD). In some embodiments, the subject has, or is suspected of having, non-exudative (dry) age-related macular degeneration. In some embodiments, the subject has, or is suspected of having, intermediate age-related macular degeneration. In some embodiments, the subject has, or is suspected of having, age-related macular degeneration with photoreceptor loss or non-exudative (dry) age-related macular degeneration with photoreceptor loss.

In some embodiments, the method further comprises separately, sequentially, or simultaneously administering a second active agent.

In some embodiments, the second active agent comprises an AREDS or AREDS 2 vitamin formula. In some embodiments, the second active agent is selected from the group consisting of: an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, or a carotenoid. In some embodiments, the second active agent is mometasone furoate, tacrolimus, quercetin, or diphenhydramine.

In some embodiments, the second active agent is flavonoid, a coumarin, a phenol or a terpenoid. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

In one aspect, the present disclosure provides a method for reducing the ratio of tEZA to RPE loss as determined by an OCT scan in a mammalian subject having, or suspected of having, age-related macular degeneration (AMD), comprising administering elamipretide to the subject for a period of at least 12 weeks. In some embodiments, elamipretide is administered subcutaneously. In some embodiments, elamipretide is administered once daily at 40 mg/dose. In some embodiments, the administration of elamipretide reduces the ratio of tEZA to RPE loss until it approaches 1. In some embodiments, the subject has, or is suspected of having, geographic atrophy (GA) secondary to age-related macular degeneration (AMD). In some embodiments, the subject has, or is suspected of having, non-exudative (dry) age-related macular degeneration. In some embodiments, the subject has, or is suspected of having, intermediate age-related macular degeneration. In some embodiments, the subject has, or is suspected of having, age-related macular degeneration with photoreceptor loss or non-exudative (dry) age-related macular degeneration with photoreceptor loss.

In some embodiments, the method further comprises separately, sequentially, or simultaneously administering a second active agent.

In some embodiments, the second active agent comprises an AREDS or AREDS 2 vitamin formula. In some embodiments, the second active agent is selected from the group consisting of: an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, or a carotenoid. In some embodiments, the second active agent is mometasone furoate, tacrolimus, quercetin, or diphenhydramine.

In some embodiments, the second active agent is flavonoid, a coumarin, a phenol or a terpenoid. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

In one aspect, the present disclosure provides a method for protecting against photoreceptor loss in a mammalian subject having, or suspected of having, age-related macular degeneration (AMD), comprising administering elamipretide to the subject for a period of at least 12 weeks. In some embodiments, elamipretide is administered to the subject for at least 3 months, at least 6 months, at least 9 months, at least 12 months, at least 18 months, at least 24 months or at least 48 months.

In some embodiments, protection against photoreceptor loss is determined by measuring the difference percentage of tEZa between two time points, each as determined by examination or an OCT scan or scans, between a treated individual or group of treated individuals as compared with an untreated individual or untreated group individuals.

In some embodiments, the elamipretide is administered subcutaneously once daily at 40 mg/dose.

In some embodiments, the subject has, or is suspected of having, geographic atrophy (GA) secondary to age-related macular degeneration (AMD). In some embodiments, the subject has, or is suspected of having, non-exudative (dry) age-related macular degeneration. In some embodiments, the subject has, or is suspected of having, intermediate age-related macular degeneration. In some embodiments, the subject has, or is suspected of having, age-related macular degeneration with photoreceptor loss or non-exudative (dry) age-related macular degeneration with photoreceptor loss.

In some embodiments, the method further comprises separately, sequentially, or simultaneously administering a second active agent.

In some embodiments, the second active agent comprises an AREDS or AREDS 2 vitamin formula. In some embodiments, the second active agent is selected from the group consisting of: an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, or a carotenoid. In some embodiments, the second active agent is mometasone furoate, tacrolimus, quercetin, or diphenhydramine.

In some embodiments, the second active agent is flavonoid, a coumarin, a phenol or a terpenoid. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

### BRIEF DESCRIPTION OF THE DRAWINGS

FIG. 1 is a schematic of the clinical study design as described in Example 1.

FIG. 2A is a bar graph showing the mean change in LL-BCVA. FIG. 2B is a bar graph showing the mean change in progression of GA secondary to AMD in subjects examined in the study.

FIG. 3 is a bar graph showing elamipretide mediated a categorical improvement in LL BCVA. The mITT population was used for the analysis, placebo n=48 and elamipretide n=82. Statistical analysis showing nominal significance levels.

FIG. 4A is a chart showing elamipretide reduces total EZ attenuation. FIG. 4B is a chart showing elamipretide reduces partial EZ attenuation. Least Square (LS) means estimated from a mixed-effects model for repeated measures (MMRM). The mITT population was used for the analysis, for total EZ attenuation (tEZa), placebo n=50 and 42 for 24 and 48 weeks, respectively while elamipretide n=89 and 71, respectively. From partial EZ attenuation (pEZa), placebo n=50 and 42, for 24 and 48 weeks, respectively while elamipretide n=89 and 71, respectively. Statistical analysis showing nominal “p values.”

FIG. 5 are representative ellipsoid zone integrity maps. Areas of dark gray represent total EZ attenuation and areas of black represent partial EZ attenuation. Maps were matched for degree of baseline tEZ attenuation and growth from baseline to week 48 in exemplary patients of the placebo treatment and elamipretide treatment arms are illustrated.

FIG. 6 are representative ellipsoid zone integrity maps. Areas of dark gray total EZ attenuation and areas of black represent partial EZ attenuation. Maps were matched for degree of baseline tEZ attenuation and growth from baseline to week 48 in exemplary patients of the placebo treatment and elamipretide treatment arms are illustrated.

FIGS. 7A and 7B are charts showing elamipretide reduced progression of EZ-GA gap. The total randomized population was used for the analysis. Placebo n=59, 52, and 41 for baseline, 24, and 48 weeks, respectively, while elamipretide n=115, 83, and 73, respectively. Least Square (LS) means estimated from a mixed-effects model for repeated measures (MMRM) is used in reporting change from baseline.

FIG. 8 is a graphical representation of elamipretide induced attenuation of progression of overall retinal damage. The inner ring of each band represents the baseline area for each parameter (black (innermost band) for geographic atrophy (GA); light gray (middle band) for total EZ attenuation (tEZa); and dotted (outermost ring) for partial EZ attenuation) and the outer ring of each band shows growth over the 48-week study period.

### DETAILED DESCRIPTION

It is to be appreciated that certain aspects, modes, embodiments, variations and features of the invention are described below in various levels of detail in order to provide a substantial understanding of the present invention.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, NY, 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively.

The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administration” of an agent, drug, or peptide to a subject includes any route of introducing or delivering to a subject a compound to perform its intended function. Administration can be conducted by any suitable route, including orally, intraocularly, intranasally, parenterally (intravenously, intramuscularly, intraperitoneally, or subcutaneously), or topically. Administration includes self-administration, the administration by another or administration by a device.

As used herein, the term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, “area of photoreceptor loss” refers to the area (in mm²) of the macular where the EZ-RPE thickness equals 0 μm on en-face map and is synonymous with the term “area of total EZ attenuation (tEZa)”.

As used herein, the term “area of total EZ attenuation” or “area of total ellipsoid zone attenuation” refers to the area (in mm²) of the macular where the EZ-RPE thickness equals 0 μm on en-face map, generally as determined by analysis of an optical coherence tomography (OCT) scan.

As used herein, the term “baseline” refers to the first study visit with a subject after determining that they meet the criteria for a certain clinical trial through the screening process. The baseline is considered the date (often referred to as Day 1 of the study or t=0) and time of initiation of the study for that subject with respect to the study protocol and the associated data collected for that subject at that first study visit.

As used herein “center 1-mm EZ-RPE thickness” refers to the thickness of the combined EZ-RPE layers at 1 mm diameter surrounding the foveal center.

As used herein “central 1 mm of the EZ-RPE” refers to the combined EZ and RPE layers at 1 mm diameter surrounding the foveal center.

As used herein, the term “effective amount” refers to a quantity of a therapeutic agent sufficient to achieve a desired therapeutic and/or prophylactic effect, e.g., an amount which results in the prevention of, or a decrease in, the signs or symptoms (e.g., deterioration of vision or photoreceptor loss) associated with the disease to be treated (e.g., AMD). The amount of a therapeutic agent administered to the subject will depend on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds/agents. In the methods described herein, elamipretide may be administered to a subject having one or more signs or symptoms of AMID. For example, a “therapeutically effective amount” of elamipretide is meant levels in which the physiological effects of AMD are, at a minimum, ameliorated (e.g., slowing the rate of growth of photoreceptor loss over time).

As used herein, “elamipretide” refers to the tetrapeptide with the amino acid sequence: H-D-Arg-2′,6′Dmt-Lys-Phe-NH₂, where 2′,6′-Dmt is the amino acid 2′,6′-dimethyltyrosine. Elamipretide has the structure:

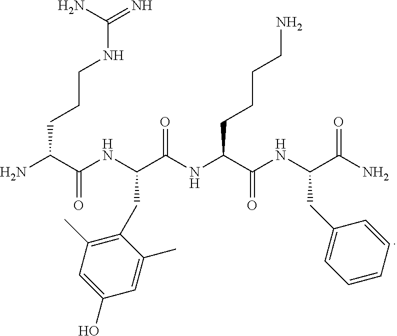 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

Elamipretide is also referred to in the scientific literature as SS-31, bendavia and MTP-131. Elamipretide is typically administered as the pharmaceutically acceptable salt, such as a tris-HCl salt having the structure:

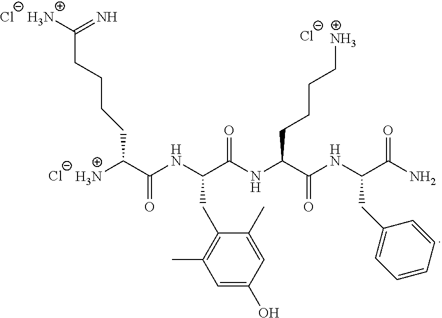 [SMILES: [Cl-] ; Cc1cc(O)cc(C)c1C[C@H](NC(=O)[C@H]([NH3+])CCCCC(=N)[NH3+])C(=O)N[C@@H](CCCC[NH3+])C(=O)N[C@@H](Cc1ccccc1)C(N)=O ; [Cl-] ; [Cl-]]

Elamipretide is commonly administered as in its pharmaceutically acceptable salt form rather than as a free-base. Whenever the term elamipretide is used herein, its use is intended to also encompass all possible pharmaceutically acceptable salts thereof, unless the context of its use is clearly contradictory to such an interpretation.

As used herein “ellipsoid zone” or “EZ” refers to the mitochondria-rich hyper-reflective outer retinal band (as seen in OCT) located just above the retinal pigment epithelium or RPE.

As used herein “en-face map” refers to an image derived using, for example, spectral domain optical coherence tomography (SD-OCT). Such en-face map generally represents a two dimensional (e.g., 6 mm×6 mm) image taken of the subject's eye.

As used herein “EZ attenuation area” refers to the area of attenuated ellipsoid zone expressed in mm².

As used herein “ellipsoid zone integrity” or “EZ integrity” refers to the state of the organization or arrangement and size of photoreceptor outer segments of the macula scan area of an individual eye in a disease state as compared to a non-diseased state.

As used herein “EZ-RPE thickness” refers to thickness of the combined ellipsoid zone (EZ) and retinal pigment epithelium (RPE).

As used herein “fovea” refers to the small depression in the retina of the eye where: (i) retinal cones are particularly concentrated; and (ii) the center of the field of vision is focused; and (iii) visual acuity is highest.

As used herein “foveal center” refers to the small, flat spot located exactly in the center of the posterior portion of the retina.

As used herein “GA area” refers to the total area (in mm²) of geographic atrophy within an individual macula of a subject's eye, which total area can be determined by examination of an en-face map for evidence of loss of photoreceptors, retinal pigment epithelium (RPE), and underlying choriocapillaris.

As used herein “geographic atrophy” or “GA” refers to a chronic progressive degeneration of the macula, as part of late-stage age-related macular degeneration (AMD), wherein geographic atrophy (GA) is defined by the presence of sharply demarcated atrophic lesions of the outer retina, resulting from loss of photoreceptors, retinal pigment epithelium (RPE), and underlying choriocapillaris.

As used herein “macular percentage of partial EZ attenuation” or “macular percentage of pEZa,” “percentage of partial EZ attenuation,” “percentage of pEZa,” “area percent of partial EZ attenuation,” or “area percent of pEZa” refers to the percentage of the macular area where the EZ-RPE thickness is ≤20 μm on en-face map (typically determined with respect to the area (in mm²) where the EZ-RPE thickness is ≤20 m divided by the total area (in mm²) of the entire en-face map to derive the percentage).

As used herein “macular percentage of total EZ attenuation” or “macular percentage of tEZa,” “percentage of total EZ attenuation,” “percentage of tEZa,” “area percent of total EZ attenuation,” or “area percent of tEZa” refers to the percentage of the macular area where the EZ-RPE thickness is 0 μm on en-face map (typically determined with respect to the area (in mm²) where the EZ-RPE thickness is 0 μm on en-face map divided by the total area (in mm²) of the entire en-face map to derive the percentage).

As used herein “optical coherence tomography” or “OCT” refers to a noninvasive imaging modality using a beam of light (i.e., light interference) to rapidly scan the eye to generate direct cross-sectional image of a subject's retina and optic nerve suitable to produce measurements of the thickness/volume of individual layers/structures of the eye. There are several types of OCT that can be used to produce a suitable OCT scan for the practice of this disclosure, including, but not limited to, time-domain OCT (i.e., TD-OCT), spectral domain OCT (i.e., SD-OCT) and swept-source (SS-OCT). If not otherwise specified, when used herein, a general reference to “optical coherence tomography” or “OCT” refers to all of the above forms of optical coherence tomography and any other form of optical coherence tomography that can be used for the examination of a subject's/patient's eyes.

As used herein “outer nuclear layer” or “ONL” refers to the outer-most layer of the retina, which contains the cell bodies of rod and cone photoreceptors.

As used herein “outer retinal parameters” refers to the outer nuclear layer (ONL) to RPE thickness.

As used herein “partial EZ attenuation” or “pEZa” refers to the portion of the macular tissue where the EZ-RPE thickness is ≤20 μm.

As used herein, “percent photoreceptor loss” refers to the percentage of the macular scan area where EZ-RPE thickness of 0 μm on en-face map (synonymous with percentage of tEZa).

As used herein, the term “pharmaceutically acceptable salt” refers to a salt of a therapeutically active compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds of the present application may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology.

As used herein, the terms “polypeptide”, “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, “prevention” or “preventing” of a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein “retinal pigment epithelium” or “RPE” refers to the single layer of tightly joined pigmented cells just outside the neurosensory retina that nourishes retinal visual cells and forms a barrier between the retina and underlying choroid.

As used herein, the term “separate” refers to an administration of at least two therapeutic agents at the same time or at substantially the same time by different routes.

As used herein, the term “sequential” refers to administration of at least two different therapeutic agents at different times, the administration route being identical or different. More particularly, sequential use refers to the whole administration of one of the at least two different therapeutic agents before administration of the other or others commences. It is thus possible to administer one of the therapeutic agents over several minutes, hours, or days before administering the other active ingredient or ingredients. There is no simultaneous treatment in this case.

As used herein, the term “simultaneous” refers to the administration of at least two different therapeutic agents by the same route and at the same time or at substantially the same time.

As used herein, the terms “subject” and “patient” are used interchangeably.

As used herein “sub-RPE anatomical metrics” refers to refers to anatomical measurements and features of the subretinal pigment epithelium.

As used herein, the terms “treating” or “treatment” or “alleviation” refers to both therapeutic treatment and prophylactic or preventative measures, wherein the object is to prevent or slow down (lessen) the targeted pathologic condition or disorder (e.g., loss of vision or loss of photoreceptors). A subject is successfully “treated” for the condition if, after receiving a therapeutic amount of a therapeutic agent (e.g., elamipretide) according to the methods described herein, the subject shows observable and/or measurable reduction in or absence of one or more signs and symptoms of the condition (e.g., loss of vision or loss of photoreceptors). It is also to be appreciated that the various modes of treatment or prevention of medical conditions as described are intended to mean “substantial,” which includes total but also less than total treatment or prevention, and wherein some biologically or medically relevant result is achieved.

As used herein “total EZ attenuation” or “tEZa” refers to the portion of the tissue of the macular where the EZ-RPE thickness is 0 μm.

### Age-Related Macular Degeneration

The pathophysiology of AMD is thought to involve multiple converging pathways, including complement activation, lipid metabolism, and mitochondrial injury (Tan et al., Redox Biology, 2020). Because of the multifactorial etiology involved, effective disease management may require treatment of multiple targets that may differ across various stages of disease (Handa et al., Nature Communications, 2019). Although the multi-faceted pathophysiology of AMD is complex and not fully understood, investigations confirm that the root cause of irreversible vision loss is photoreceptor death. Therefore, protecting photoreceptors from damage and delaying their degeneration are key to the successful treatment of clinical symptoms and disease progression (Scholl et al., Ophthalmic Res, 2021).

Photoreceptor dysfunction/loss/death in AMD can be quantified by changes in the ellipsoid zone (EZ), which is a mitochondria-rich tissue layer containing photoreceptors, and the associated loss (i.e., death) of photoreceptor cells leading to (and correlated with) progressive loss of visual function (Abraham, et al., Ophthamol Ret, 2022; Sarici, et al., Ophthamol. Surg Lasers Imag. Ret, 2022; Itoh, et al., Br J Ophthamol, 2015). These observed EZ changes are typically associated with the eventual loss (i.e., death) of sections of the retinal pigment epithelium (RPE) and the underlying choriocapillaris leading to areas of GA. Notably, EZ loss/death or attenuation has been shown to predict areas of GA, as well as precede its onset by two to three years (Pasricha et al., Ophthalmol Retina, 2021, Sarici Ophthalmic Surg Lasers Imaging Retina. 2022), suggesting that photoreceptor dysfunction/loss/death may precede the loss of underlying RPE. EZ loss/death has been associated with GA progression (Abraham et al., ASRS, 2022), and there is emerging data suggesting that attenuation (e.g., reduction in rate) of EZ loss (i.e., death) may mitigate/slow/delay GA progression (Mai et al., Am J Ophthalmol, 2022; Ehlers, et al., Macula Society, 2022; Vogl, et al. Ophthalmol Retina, 2023). Longitudinal studies have also demonstrated that the EZ loss/death-to-GA boundary distance (EZ-GA gap) is prognostic for future GA progression rates (Pfau et al., JAMA Ophthalmol., 2020; Ehlers, et al., Macula Society, 2022). Accordingly, it has been postulated that imaging photoreceptor degeneration may serve not only as a prognostic biomarker, but as attractive clinical end points in dry AMD since they are accurate, with high prognostic validity, and have been concurrently validated in multiple structure-function correlation studies in dry AMD (Pfau et al., JAMA Ophthalmol., 2020).

Patients with dry AMD usually report or present early in the course of the disease with visual functional impairments including difficulty reading or limited vision at night or in reduced lighting conditions (Chandramohan 2016, Bressler 2003). This early loss of low luminance best corrected visual acuity (LL BCVA) occurs despite relative preservation of best-corrected visual acuity (BCVA) in the absence of foveal atrophy (Fleckenstein 2018). Low luminance visual dysfunction has been shown to be predictive of subsequent best corrected visual dysfunction in eyes with GA (Sunness 2008). LL BCVA is also significantly associated with patient-reported visual quality of life (Künzel et al., Invest Ophthalmol Vis Sci., 2020). Accordingly, the preservation of LL BCVA is an important and relevant clinical endpoint for dry AMD (Sunness 2008; Oct. 2, 2018, Type C Meeting Preliminary Comments). Notably, in the setting of intermediate AMD, degeneration of EZ-RPE thickness and reflectivity is strongly associated with loss of mesopic and scotopic light sensitivity (Pfau et al., JAMA Ophthalmol., 2020) which in turn is associated with LL BCVA decline (Pondorfer et al., PLOS One, 2020).

### Prophylactic and Therapeutic Uses of Elamipretide.

Elamipretide can be useful in the prevention or treatment of AMD, GA secondary to AMD, non-exudative (dry) age-related macular degeneration, exudative (wet) age-related macular degeneration, intermediate age-related macular degeneration, age-related macular degeneration with photoreceptor loss or non-exudative (dry) age-related macular degeneration with photoreceptor loss. Specifically, the present disclosure provides for both prophylactic and therapeutic methods of treating a subject having or suspected of having AMD, GA secondary to AMD, non-exudative (dry) age-related macular degeneration, exudative (wet) age-related macular degeneration, intermediate age-related macular degeneration, age-related macular degeneration with photoreceptor loss or non-exudative (dry) age-related macular degeneration with photoreceptor loss. Accordingly, the present methods provide for the treatment, prevention or delay (slowing) of the progression of AMID in a mammalian subject by administering an effective amount of elamipretide to a subject in need thereof. For example, a subject can be administered elamipretide containing compositions in an effort to improve one or more of the factors contributing to AMD or to delay the effects of deterioration/loss of vision and/or the tissue/photoreceptors that lead to AMD, GA secondary to AMD, non-exudative (dry) age-related macular degeneration, exudative (wet) age-related macular degeneration, intermediate age-related macular degeneration, age-related macular degeneration with photoreceptor loss or non-exudative (dry) age-related macular degeneration with photoreceptor loss.

One aspect of the technology provides for a method of treating, preventing or delaying (slowing) the progress of AMID, GA secondary to AMD, non-exudative (dry) age-related macular degeneration, exudative (wet) age-related macular degeneration, intermediate age-related macular degeneration, age-related macular degeneration with photoreceptor loss or non-exudative (dry) age-related macular degeneration with photoreceptor loss in a mammalian subject for therapeutic purposes. In therapeutic applications, compounds (e.g., elamipretide), compositions or medicaments (e.g., formulations comprising elamipretide) are administered to a subject suspected of, or already suffering from AMD in an amount sufficient to at least partially arrest (i.e., delay or slow), the signs or symptoms of the disease, including its complications and intermediate pathological phenotypes in development of the disease. As such, the disclosure provides methods of treating an individual afflicted with AMD. Furthermore, this disclosure provided methods for preventing or delaying (slowing) the progression of AMD (including wet- or dry-AMD) as well as treating, preventing or delaying (slowing) the progression of GA secondary to AMD, non-exudative (dry) age-related macular degeneration, intermediate age-related macular degeneration, age-related macular degeneration with photoreceptor loss or non-exudative (dry) age-related macular degeneration with photoreceptor loss.

In one aspect, the present disclosure provides a method for treating, preventing or delaying (slowing) the progression of age-related macular degeneration (AMD) in a mammalian subject in need thereof, comprising the steps of: a) selecting subjects diagnosed as having or suspected of having AMD; b) examining one or both eyes of the subject using optical coherence tomography (OCT) to produce a first OCT scan for each eye examined; c) analyzing each OCT scan to determine: (i) the area percent of partial EZ attenuation (pEZa); (ii) the area percent of total EZ attenuation (tEZa); and/or (iii) the center 1-mm ellipsoid zone-retinal pigment epithelium (EZ-RPE) thickness; and d) administering an effective amount of elamipretide to subjects presenting with; (i) an area percent of partial EZ attenuation (pEZa) greater than zero and less than 100 percent; (ii) an area percent of total EZ attenuation (tEZa) is greater than zero and less than 100 percent; and/or (iii) a center 1-mm EZ-RPE thickness is greater than 20 μm.

In some embodiments, the elamipretide is administered to the subject on a daily, weekly, or monthly basis.

In some embodiments, the area percent of tEZa is greater than zero and less than 50 percent. In some embodiments, the area percent of tEZa is greater than zero and less than 30 percent. In some embodiments, the area percent of tEZa is greater than zero and less than 20 percent. In some embodiments, the subject has high-risk drusen (HRD).

In some embodiments, analyzing the first OCT scan comprises evaluating higher-order OCT features using automated machine-learning augmented multilayer retinal segmentation optionally with expert manual reader verification.

In some embodiments, the subject is also evaluated using a visual acuity test and at baseline has a best corrected visual acuity (BCVA) in at least one eye of greater than or equal to 55 letters and a low luminance best corrected visual acuity (LL-BCVA) deficit of at least 5 letters.

In some embodiments, administration of elamipretide leads to at least a 2 line gain (i.e., at least 10 letters) in LL BCVA from baseline in the subject after at least one year of elamipretide administration. In some embodiments, administration of elamipretide leads to at least a 3 line gain (i.e., at least 15 letters) in LL BCVA from baseline in the subject after at least one year of elamipretide administration.

In some embodiments, the method further comprises examining one or both eyes of the subject using OCT to produce an additional OCT scan for each eye examined.

In some embodiments, analyzing the additional OCT scan comprises evaluating higher-order OCT features using automated machine-learning augmented multilayer retinal segmentation optionally with expert manual reader verification.

In some embodiments, the subject is a human.

In some embodiments, the elamipretide is administered intraocularly, iontophoretically, orally, topically, systemically, intravenously, subcutaneously, or intramuscularly.

In some embodiments, the method further comprises separately, sequentially, or simultaneously administering a second active agent.

In some embodiments, the second active agent comprises an AREDS or AREDS2 vitamin formula. The AREDS vitamin formula comprises 400 IU of vitamin E, 15 mg of beta-carotene, 80 mg zinc as zinc oxide and 2 mg copper as cupric oxide. The AREDS2 vitamin formula comprises 10 mg of lutein, 2 mg of zeazanthin, 500 mg of vitamin C, 400 IU of vitamin E, 80 (or 25) mg of zinc oxide and 2 mg of cupric oxide.

In some embodiments, the second active agent is selected from the group consisting of: an antioxidant, a metal complexer, an anti-inflammatory drug, an antibiotic, and an antihistamine.

In some embodiments, the antioxidant is vitamin A, vitamin C, vitamin E, lycopene, selenium, α-lipoic acid, coenzyme Q, glutathione, or a carotenoid.

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

In some embodiments, about 5 mg to about 80 mg of elamipretide, or a pharmaceutically acceptable salt thereof, is subcutaneously administered to the injection site of the subject.

In some embodiments, about 10 mg to about 60 mg of elamipretide, or a pharmaceutically acceptable salt thereof, is subcutaneously administered to the injection site of the subject.

In some embodiments, about 20 mg to about 60 mg of elamipretide, or a pharmaceutically acceptable salt thereof, is subcutaneously administered to the injection site of the subject.

In some embodiments, about 20 mg of elamipretide, or a pharmaceutically acceptable salt thereof, is subcutaneously administered to the injection site of the subject.

In some embodiments, about 40 mg of elamipretide, or a pharmaceutically acceptable salt thereof, is subcutaneously administered to the injection site of the subject.

In some embodiments, 40 mg of elamipretide, or a pharmaceutically acceptable salt thereof, is subcutaneously administered to the injection site of the subject.

In some embodiments, about 60 mg of elamipretide, or a pharmaceutically acceptable salt thereof, is subcutaneously administered to the injection site of the subject.

In some embodiments, 60 mg of elamipretide, or a pharmaceutically acceptable salt thereof, is subcutaneously administered to the injection site of the subject.

In some embodiments, the subject presents with an area percent of partial EZ attenuation (pEZa) greater than 5 and less than 80 percent.

In some embodiments, the subject presents with a center 1-mm EZ-RPE thickness that is greater than 20 μm.

In some embodiments, the aforementioned method further provides for treating, preventing or delaying (slowing) the progression of geographic atrophy (GA) secondary to age-related macular degeneration (AMD). In some embodiments, the aforementioned method further provides for treating, preventing or delaying (slowing) the progression of non-exudative (dry) age-related macular degeneration. In some embodiments, the aforementioned method provides for treating, preventing or delaying (slowing) the progression of intermediate age-related macular degeneration. In some embodiments, the aforementioned method further provides for treating, preventing or delaying (slowing) the progression of age-related macular degeneration with photoreceptor loss or non-exudative (dry) age-related macular degeneration with photoreceptor loss. In some embodiments, the aforementioned method further provides for treating, preventing or delaying (slowing) the progression of age-related macular degeneration by slowing the rate of photoreceptor loss.

In one aspect, the present disclosure provides a method comprising: a) examining one or both eyes of a mammalian subject using optical coherence tomography (OCT) to produce a 1^{st} OCT scan for each eye examined; b) reexamining one or both eyes of the subject using optical coherence tomography (OCT) to produce a 2^{nd} OCT scan for one or both of the subject's eyes on a day that is after performing the examination according to step (a); c) optionally repeating step (b) n-times, each n^{th} reexamination occurring on a day that is after the n^{th}−1 examination, that produced an n^{th}−1 OCT scan, to thereby produce a n^{th} OCT scan for one or both eyes, where n is a whole number from 3 to 100; and d) examining the OCT scans to determine: (i) a rate of photoreceptor loss; (ii) a percent of photoreceptor loss; and/or (iii) a mean change in the macular area of photoreceptor loss; in each eye examined for the subject that has occurred from: a) the 1^{st} OCT scan to the 2^{nd} OCT scan or to any one or more of the n^{th} OCT scans; and/or b) between two later obtained OCT scans for a particular eye examined.

In some embodiments, the method further comprises prescribing the administration of elamipretide for the subject to thereby treat, prevent or delay (slow) progression of the subject's photoreceptor loss in one or both eyes. In some embodiments, the method further comprises identifying the subject as a candidate for the administration of elamipretide to thereby treat, prevent or delay (slow) progression of the subject's photoreceptor loss in one or both eyes. In some embodiments, the method further comprises administering elamipretide to the subject to thereby treat, prevent or delay (slow) progression of the subject's photoreceptor loss in one or both eyes. In some embodiments, elamipretide is administered subcutaneously to the subject once daily at 40 mg/dose.

To be clear, photoreceptor loss can be determined from the OCT scan by measuring the distance from the EZ segmentation line to the RPE segmentation line, and when that measurement equals 0 m, photoreceptors are considered to be lost (also referred to as tEZa). The area percent of photoreceptor loss is determined from the OCT scan by taking the area of photoreceptor loss (generally measured in mm²) and dividing by the macular scan area (otherwise referred to as the area (usually measured in mm2) of the en-face map; the result being also referred to as the “area percentage of tEZa”). Photoreceptor loss is determined on an eye-by-eye basis by comparing the area percent of photoreceptor loss in an earlier OCT scan for the eye of a patient and comparing the area percent of photoreceptor loss to a later performed OCT scan. Based on these analyses, it has been determined that treatment of subjects with elamipretide slows the progression of photoreceptor loss. Thus, it can be said that for subjects treated with elamipretide, photoreceptor loss will be slower (area percent of tEZa will be lower/lesser) as compared with an untreated subject or untreated control group of subjects.

In some embodiments, the 2^{nd} OCT scan is produced between 1 month and 3 months, or 3 months and 6 months, or 6 months and one year, after the 1^{st} OCT scan is produced. In some embodiments, each n^{th} OCT scan is produced between 1 month and 3 months, or 3 months and 6 months, or 6 months and one year, after the n^{th}−1 OCT scan is produced.

In some embodiments, delaying (slowing) progression of the subject's photoreceptor loss in one or both eyes correspond with delaying (slowing) progression of (wet or dry) age-related macular degeneration (AMD) in one or both of the subject's eyes. In some embodiments, delaying (slowing) progression of the subject's photoreceptor loss in one or both eyes correspond with delaying (slowing) progression of geographic atrophy (GA) secondary to age-related macular degeneration (AMD) in one or both of the subject's eyes. In some embodiments, the method further provides for treating, preventing or delaying (slowing) the progression of non-exudative (dry) age-related macular degeneration in one or both of the subject's eyes. In some embodiments, the method further provides for treating, preventing or delaying (slowing) the progression of intermediate age-related macular degeneration in one or both of the subject's eyes. In some embodiments, the method further provides for treating, preventing or delaying (slowing) the progression of age-related macular degeneration with photoreceptor loss or non-exudative (dry) age-related macular degeneration with photoreceptor loss in one or both of the subject's eyes. In some embodiments, the method further provides for treating, preventing or delaying (slowing) the progression of age-related macular degeneration by slowing the rate of photoreceptor loss in one or both of the subject's eyes.

In some embodiments, the subject is a human.

In some embodiments, the elamipretide is administered intraocularly, iontophoretically, orally, topically, systemically, intravenously, subcutaneously, or intramuscularly.

In some embodiments, the method further comprises separately, sequentially, or simultaneously administering a second active agent.

In some embodiments, the second active agent comprises an AREDS or AREDS2 vitamin formula. The AREDS vitamin formula comprises 400 IU of vitamin E, 15 mg of beta-carotene, 80 mg zinc as zinc oxide and 2 mg copper as cupric oxide. The AREDS2 vitamin formula comprises 10 mg of lutein, 2 mg of zeazanthin, 500 mg of vitamin C, 400 IU of vitamin E, 80 (or 25) mg of zinc oxide and 2 mg of cupric oxide.

In some embodiments, the second active agent is selected from the group consisting of: an antioxidant, a metal complexer, an anti-inflammatory drug, an antibiotic, and an antihistamine.

In some embodiments, the antioxidant is vitamin A, vitamin C, vitamin E, lycopene, selenium, α-lipoic acid, coenzyme Q, glutathione, or a carotenoid.

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

In some embodiments, about 5 mg to about 80 mg of elamipretide, or a pharmaceutically acceptable salt thereof, is subcutaneously administered to the injection site of the subject.

In some embodiments, about 10 mg to about 60 mg of elamipretide, or a pharmaceutically acceptable salt thereof, is subcutaneously administered to the injection site of the subject.

In some embodiments, about 20 mg to about 60 mg of elamipretide, or a pharmaceutically acceptable salt thereof, is subcutaneously administered to the injection site of the subject.

In some embodiments, about 20 mg of elamipretide, or a pharmaceutically acceptable salt thereof, is subcutaneously administered to the injection site of the subject.

In some embodiments, about 40 mg of elamipretide, or a pharmaceutically acceptable salt thereof, is subcutaneously administered to the injection site of the subject.

In some embodiments, 40 mg of elamipretide, or a pharmaceutically acceptable salt thereof, is subcutaneously administered to the injection site of the subject.

In some embodiments, about 60 mg of elamipretide, or a pharmaceutically acceptable salt thereof, is subcutaneously administered to the injection site of the subject.

In some embodiments, 60 mg of elamipretide, or a pharmaceutically acceptable salt thereof, is subcutaneously administered to the injection site of the subject.

In some embodiments, the subject presents with an area percent of partial EZ attenuation (pEZa) greater than 5 and less than 80 percent.

In some embodiments, the subject presents with a center 1-mm EZ-RPE thickness that is greater than 20 μm.

In one aspect, the present disclosure provides a method comprising: a) examining one or both eyes of a mammalian subject using a visual acuity test to determine the subject's 1^{st} low luminance best corrected visual acuity (1^{st} LL BCVA), collectively and/or on an eye-by-eye basis; b) reexamining one or both eyes of the subject using a visual acuity test, to thereby determine the subject's 2^{nd} low luminance best corrected visual acuity (2^{nd} LL BCVA), collectively and/or on an eye-by-eye basis, on a day that is after performing the examination according to step (a); c) optionally repeating step (b) n-times, to thereby determine the subject's n^{th} low luminance best corrected visual acuity (n^{th} LL BCVA), collectively and/or on an eye-by-eye basis, each n^{th} reexamination occurring on a day that is after the n^{th}−1 examination, where n is a whole number from 3 to 100; and d) determining whether the subject has lost or gained one or more letters and/or one or more lines in LL BCVA, collectively or on an eye-by-eye basis, (a) from the 1^{st} LL BCVA determination to the 2^{nd} LL BCVA determination or any one or more of the n^{th} LL BCVA determinations; and/or b) between two later LL BCVA determinations.

In some embodiments, the method further comprises prescribing the administration of elamipretide for the subject to thereby treat, prevent or delay (slow) progression of the subject's loss in LL BCVA, collectively or on an eye-by-eye basis. In some embodiments, the method further comprises identifying the subject as a candidate for the administration of elamipretide to thereby treat, prevent or delay (slow) progression of the subject's loss in LL BCVA, collectively or on an eye-by-eye basis. In some embodiments, the method further comprises administering elamipretide to the subject to thereby treat, prevent or delay (slow) progression of the subject's loss in LL BCVA, collectively or on an eye-by-eye basis. In some embodiments, elamipretide is administered subcutaneously to the subject once daily at 40 mg/dose.

In some embodiments of the method, the 2^{nd} LL BCVA determination is performed between 1 month and 3 months, or 3 months and 6 months, or 6 months and one year, after the 1^{st} LL BCVA determination is performed. In some embodiments, each n^{th} LL BCVA determination is performed between 1 month and 3 months, or 3 months and 6 months, or 6 months and one year, after the n^{th}-LL BCVA determination is performed. In some embodiments, the LL BCVA for a subject is determined on an annual basis.

In some embodiments, delaying (slowing) progression of the subject's loss in LL BCVA in one or both eyes corresponds with delaying (slowing) progression of (wet or dry) age-related macular degeneration (AMD) in one or both of the subject's eyes. In some embodiments, delaying (slowing) progression of the subject's loss in LL BCVA in one or both eyes corresponds with delaying (slowing) progression of geographic atrophy (GA) secondary to age-related macular degeneration (AMD) in one or both of the subject's eyes. In some embodiments, delaying (slowing) progression of the subject's loss in LL BCVA in one or both eyes corresponds with delaying (slowing) progression of non-exudative (dry) age-related macular degeneration in one or both of the subject's eyes. In some embodiments, delaying (slowing) progression of the subject's loss in LL BCVA in one or both eyes correspond with delaying (slowing) progression of intermediate age-related macular degeneration in one or both of the subject's eyes. In some embodiments, delaying (slowing) progression of the subject's loss in LL BCVA in one or both eyes corresponds with delaying (slowing) progression of age-related macular degeneration with photoreceptor loss or non-exudative (dry) age-related macular degeneration with photoreceptor loss in one or both of the subject's eyes. In some embodiments, delaying (slowing) progression of the subject's loss in LL BCVA in one or both eyes corresponds with delaying (slowing) progression of age-related macular degeneration by slowing the rate of photoreceptor loss in one or both of the subject's eyes. In some embodiments, delaying (slowing) progression of the subject's loss in LL BCVA in one or both eyes corresponds with delaying (slowing) progression of photoreceptor loss in one or both of the subject's eyes.

In some embodiments, the subject is a human.

In some embodiments, the elamipretide is administered intraocularly, iontophoretically, orally, topically, systemically, intravenously, subcutaneously, or intramuscularly.

In some embodiments, the method further comprises separately, sequentially, or simultaneously administering a second active agent.

In some embodiments, the second active agent comprises an AREDS or AREDS2 vitamin formula. The AREDS vitamin formula comprises 400 IU of vitamin E, 15 mg of beta-carotene, 80 mg zinc as zinc oxide and 2 mg copper as cupric oxide. The AREDS2 vitamin formula comprises 10 mg of lutein, 2 mg of zeazanthin, 500 mg of vitamin C, 400 IU of vitamin E, 80 (or 25) mg of zinc oxide and 2 mg of cupric oxide.

In some embodiments, the second active agent is selected from the group consisting of: an antioxidant, a metal complexer, an anti-inflammatory drug, an antibiotic, and an antihistamine.

In some embodiments, the antioxidant is vitamin A, vitamin C, vitamin E, lycopene, selenium, α-lipoic acid, coenzyme Q, glutathione, or a carotenoid.

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

In some embodiments, about 5 mg to about 80 mg of elamipretide, or a pharmaceutically acceptable salt thereof, is subcutaneously administered to the injection site of the subject.

In some embodiments, about 10 mg to about 60 mg of elamipretide, or a pharmaceutically acceptable salt thereof, is subcutaneously administered to the injection site of the subject.

In some embodiments, about 20 mg to about 60 mg of elamipretide, or a pharmaceutically acceptable salt thereof, is subcutaneously administered to the injection site of the subject.

In some embodiments, about 20 mg of elamipretide, or a pharmaceutically acceptable salt thereof, is subcutaneously administered to the injection site of the subject.

In some embodiments, about 40 mg of elamipretide, or a pharmaceutically acceptable salt thereof, is subcutaneously administered to the injection site of the subject.

In some embodiments, 40 mg of elamipretide, or a pharmaceutically acceptable salt thereof, is subcutaneously administered to the injection site of the subject.

In some embodiments, about 60 mg of elamipretide, or a pharmaceutically acceptable salt thereof, is subcutaneously administered to the injection site of the subject.

In some embodiments, 60 mg of elamipretide, or a pharmaceutically acceptable salt thereof, is subcutaneously administered to the injection site of the subject.

In some embodiments, the subject presents with an area percent of partial EZ attenuation (pEZa) greater than 5 and less than 80 percent.

In one aspect, the present disclosure provides a method for improving the low luminance best corrected visual acuity (LL BCVA) by 2 or more lines, collectively or on an eye-by-eye basis, in a mammalian subject having, or suspected of having, age-related macular degeneration (AMD), comprising administering elamipretide to the subject for a period of at least 12 weeks. In some embodiments, the elamipretide is administered subcutaneously. In some embodiments, the elamipretide is administered once daily at 40 mg/dose. In some embodiments, the subject's low luminance best corrected visual acuity (LL BCVA) is increased by 3 or more lines, by 4 or more lines or by 5 or more lines in one or both of the subject's eyes. In some embodiments, the subject has been diagnosed as having geographic atrophy (GA) secondary to age-related macular degeneration (AMD). In some embodiments, the subject has, or is suspected of having, non-exudative (dry) age-related macular degeneration. In some embodiments, the subject has, or is suspected of having, intermediate age-related macular degeneration. In some embodiments, the subject has, or is suspected of having, age-related macular degeneration with photoreceptor loss or non-exudative (dry) age-related macular degeneration with photoreceptor loss.

In some embodiments, the subject is a human.

In some embodiments, the elamipretide is administered intraocularly, iontophoretically, orally, topically, systemically, intravenously, subcutaneously, or intramuscularly.

In some embodiments, the method further comprises separately, sequentially, or simultaneously administering a second active agent.

In some embodiments, the second active agent comprises an AREDS or AREDS2 vitamin formula. The AREDS vitamin formula comprises 400 IU of vitamin E, 15 mg of beta-carotene, 80 mg zinc as zinc oxide and 2 mg copper as cupric oxide. The AREDS2 vitamin formula comprises 10 mg of lutein, 2 mg of zeazanthin, 500 mg of vitamin C, 400 IU of vitamin E, 80 (or 25) mg of zinc oxide and 2 mg of cupric oxide.

In some embodiments, the second active agent is selected from the group consisting of: an antioxidant, a metal complexer, an anti-inflammatory drug, an antibiotic, and an antihistamine.

In some embodiments, the antioxidant is vitamin A, vitamin C, vitamin E, lycopene, selenium, α-lipoic acid, coenzyme Q, glutathione, or a carotenoid.

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

In some embodiments, about 5 mg to about 80 mg of elamipretide, or a pharmaceutically acceptable salt thereof, is subcutaneously administered to the injection site of the subject.

In some embodiments, about 10 mg to about 60 mg of elamipretide, or a pharmaceutically acceptable salt thereof, is subcutaneously administered to the injection site of the subject.

In some embodiments, about 20 mg to about 60 mg of elamipretide, or a pharmaceutically acceptable salt thereof, is subcutaneously administered to the injection site of the subject.

In some embodiments, about 20 mg of elamipretide, or a pharmaceutically acceptable salt thereof, is subcutaneously administered to the injection site of the subject.

In some embodiments, about 40 mg of elamipretide, or a pharmaceutically acceptable salt thereof, is subcutaneously administered to the injection site of the subject.

In some embodiments, 40 mg of elamipretide, or a pharmaceutically acceptable salt thereof, is subcutaneously administered to the injection site of the subject.

In some embodiments, about 60 mg of elamipretide, or a pharmaceutically acceptable salt thereof, is subcutaneously administered to the injection site of the subject.

In some embodiments, 60 mg of elamipretide, or a pharmaceutically acceptable salt thereof, is subcutaneously administered to the injection site of the subject.

In some embodiments, the subject presents with an area percent of partial EZ attenuation (pEZa) greater than 5 and less than 80 percent.

In one aspect, the present disclosure provides a method for reducing the ratio of tEZA to RPE loss as determined by an OCT scan in a mammalian subject having, or suspected of having, age-related macular degeneration (AMD), comprising administering elamipretide to the subject for a period of at least 12 weeks. In some embodiments, elamipretide is administered subcutaneously. In some embodiments, elamipretide is administered once daily at 40 mg/dose. In some embodiments, the administration of elamipretide reduces the ratio of tEZA to RPE loss until it approaches 1. In some embodiments, the subject has, or is suspected of having, geographic atrophy (GA) secondary to age-related macular degeneration (AMD). In some embodiments, the subject has, or is suspected of having, non-exudative (dry) age-related macular degeneration. In some embodiments, the subject has, or is suspected of having, intermediate age-related macular degeneration. In some embodiments, the subject has, or is suspected of having, age-related macular degeneration with photoreceptor loss or non-exudative (dry) age-related macular degeneration with photoreceptor loss.

In some embodiments, the method further comprises separately, sequentially, or simultaneously administering a second active agent.

In some embodiments, the second active agent comprises an AREDS or AREDS 2 vitamin formula. In some embodiments, the second active agent is selected from the group consisting of: an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, or a carotenoid. In some embodiments, the second active agent is mometasone furoate, tacrolimus, quercetin, or diphenhydramine.

In some embodiments, the second active agent is flavonoid, a coumarin, a phenol or a terpenoid. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

In one aspect, the present disclosure provides a method for protecting against photoreceptor loss in a mammalian subject having, or suspected of having, age-related macular degeneration (AMD), comprising administering elamipretide to the subject for a period of at least 12 weeks. In some embodiments, elamipretide is administered to the subject for at least 3 months, at least 6 months, at least 9 months, at least 12 months, at least 18 months, at least 24 months or at least 48 months.

In some embodiments, protection against photoreceptor loss is determined by measuring the difference percentage of tEZa between two time points, each as determined by examination or an OCT scan or scans, between a treated individual or group of treated individuals as compared with an untreated individual or untreated group individuals.

In some embodiments, the elamipretide is administered subcutaneously once daily at 40 mg/dose.

In some embodiments, the subject has, or is suspected of having, geographic atrophy (GA) secondary to age-related macular degeneration (AMD). In some embodiments, the subject has, or is suspected of having, non-exudative (dry) age-related macular degeneration. In some embodiments, the subject has, or is suspected of having, intermediate age-related macular degeneration. In some embodiments, the subject has, or is suspected of having, age-related macular degeneration with photoreceptor loss or non-exudative (dry) age-related macular degeneration with photoreceptor loss.

In some embodiments, the method further comprises separately, sequentially, or simultaneously administering a second active agent.

In some embodiments, the second active agent comprises an AREDS or AREDS 2 vitamin formula. In some embodiments, the second active agent is selected from the group consisting of: an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, or a carotenoid. In some embodiments, the second active agent is mometasone furoate, tacrolimus, quercetin, or diphenhydramine.

In some embodiments, the second active agent is flavonoid, a coumarin, a phenol or a terpenoid. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

Determination of the Biological Effect of Elamipretide. In various embodiments, suitable in vitro or in vivo assays are performed to determine the effect of elamipretide and whether its administration is indicated for treatment. In various embodiments, in vitro assays can be performed with representative cells of the type(s) involved in the subject's disorder, to determine if elamipretide exerts the desired effect upon the cell type(s). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

### Modes of Administration and Effective Dosages

Any method known to those in the art for contacting a cell, organ or tissue with a peptide may be employed. Suitable methods include in vitro, ex vivo, or in vivo methods. In vivo methods typically include the administration of elamipretide, such as those described above, to a mammal, preferably a human. When used in vivo for therapy, elamipretide is administered to the subject in effective amounts (i.e., amounts that have desired therapeutic effect). The dose and dosage regimen will depend upon the degree of the AMD in the subject, the characteristics of elamipretide, e.g., its therapeutic index, the subject, and the subject's history.

The effective amount may be determined during pre-clinical trials and clinical trials by methods familiar to physicians and clinicians. An effective amount of elamipretide useful in the methods disclosed herein, preferably in a pharmaceutical composition, may be administered to a mammal in need thereof by any of a number of well-known methods for administering pharmaceutical compounds. In some embodiments, the peptide may be administered intraocularly, iontophoretically, orally, topically, systemically, intravenously, subcutaneously, or intramuscularly.

Elamipretide can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For the convenience of the patient or treating physician, the dosing formulation can be provided in a kit containing all necessary equipment (e.g., vials of drug, vials of diluent, syringes and needles) for a treatment course.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The compositions comprising elamipretide can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it may be desirable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

For ophthalmic applications, the therapeutic compound is formulated into solutions, suspensions, and ointments appropriate for use in the eye. For ophthalmic formulations generally, see Mitra (ed.), Ophthalmic Drug Delivery Systems, Marcel Dekker, Inc., New York, N.Y. (1993) and also Havener, W. H., Ocular Pharmacology, C.V. Mosby Co., St. Louis (1983). Ophthalmic pharmaceutical compositions may be adapted for topical administration to the eye in the form of solutions, suspensions, ointments, creams or as a solid insert.

The ophthalmic preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable level of elamipretide in the eye. Administration to the mammalian eye may be about once or twice daily.

Oral compositions generally include an inert diluent or an edible carrier. For the purpose of oral therapeutic administration, the active compound can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic protein or peptide can be formulated in a carrier system. The carrier can be a colloidal system. The colloidal system can be a liposome, a phospholipid bilayer vehicle. In one embodiment, the therapeutic peptide is encapsulated in a liposome while maintaining peptide integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34 (7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic peptide can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylacetic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods 4 (3) 201-9 (1994); and Gregoriadis, “Engineering Liposomes for Drug Delivery: Progress and Problems,” Trends Biotechnol. 13 (12):527-37 (1995). Mizguchi et al., Cancer Lett. 100:63-69 (1996), describes the use of fusogenic liposomes to deliver a protein to cells both in vivo and in vitro.

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses more accurately in humans. Levels in plasma may be measured, for example, by high performance liquid chromatography.

Typically, an effective amount of elamipretide, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of peptide ranges from 0.1-10,000 micrograms per kg body weight. In one embodiment, elamipretide concentrations in a carrier range from 0.2 to 2000 micrograms per delivered milliliter. An exemplary treatment regime entails administration once per day or once a week. Intervals can also be irregular as indicated by measuring blood levels of glucose or insulin in the subject and adjusting dosage or administration accordingly. In therapeutic applications, a relatively high dosage at relatively short intervals is sometimes required until progression of the disease is reduced or terminated, and preferably until the subject shows partial or complete amelioration of symptoms of disease. Thereafter, the patient can be administered a prophylactic regime.

In some embodiments, a therapeutically effective amount of elamipretide may be defined as a concentration of peptide at the target tissue of 10⁻¹¹ to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, most preferably by single daily or weekly administration, but also including continuous administration (e.g., parenteral infusion or transdermal application).

In some embodiments, the dosage of elamipretide is provided at a “low,” “mid,” or “high” dose level. In one embodiment, the low dose is provided from about 0.0001 to about 0.5 mg/kg/h, suitably from about 0.01 to about 0.1 mg/kg/h. In one embodiment, the mid-dose is provided from about 0.1 to about 1.0 mg/kg/h, suitably from about 0.1 to about 0.5 mg/kg/h. In one embodiment, the high dose is provided from about 0.5 to about 10 mg/kg/h, suitably from about 0.5 to about 2 mg/kg/h.

In some embodiments of the methods disclosed herein, about 5 mg to about 80 mg of elamipretide, or a pharmaceutically acceptable salt thereof, is subcutaneously administered to the injection site of the subject. In some embodiments of the methods disclosed herein, about 10 mg to about 60 mg of elamipretide, or a pharmaceutically acceptable salt thereof, is subcutaneously administered to the injection site of the subject. In some embodiments of the methods disclosed herein, about 20 mg to about 60 mg of elamipretide, or a pharmaceutically acceptable salt thereof, is subcutaneously administered to the injection site of the subject. In some embodiments of the methods disclosed herein, about 20 mg of elamipretide, or a pharmaceutically acceptable salt thereof, is subcutaneously administered to the injection site of the subject. In some embodiments of the methods disclosed herein, about 40 mg of elamipretide, or a pharmaceutically acceptable salt thereof, is subcutaneously administered to the injection site of the subject. In some embodiments of the methods disclosed herein, about 60 mg of elamipretide, or a pharmaceutically acceptable salt thereof, is subcutaneously administered to the injection site of the subject. In some embodiments of the methods disclosed herein, 60 mg of elamipretide, or a pharmaceutically acceptable salt thereof, is subcutaneously administered to the injection site of the subject.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In a preferred embodiment, the mammal is a human.

Combination Therapy with Elamipretide and Other Therapeutic Agents

In certain instances, it may be appropriate to administer elamipretide (or a pharmaceutically acceptable salt, ester, amide, prodrug, or solvate) in combination with another therapeutic agent (i.e., a second active agent). By way of example only, if one of the side effects experienced by a patient upon receiving elamipretide herein is inflammation, then it may be appropriate to administer an anti-inflammatory agent in combination with the initial therapeutic agent (e.g., elamipretide and an anti-inflammatory agent). Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit in the prevention or treatment of AMD. By way of example only, in a treatment for macular degeneration involving administration of elamipretide, increased therapeutic benefit may result by also providing the patient with other therapeutic agents or therapies for macular degeneration. In any case, the overall benefit experienced by the patient may simply be additive of the two therapeutic (i.e. active) agents or the patient may experience a synergistic benefit.

In some embodiments, the second active agent comprises an AREDS or AREDS2 vitamin formula. The AREDS vitamin formula comprises 400 IU of vitamin E, 15 mg of beta-carotene, 80 mg zinc as zinc oxide and 2 mg copper as cupric oxide. The AREDS2 vitamin formula comprises 10 mg of lutein, 2 mg of zeazanthin, 500 mg of vitamin C, 400 IU of vitamin E, 80 (or 25) mg of zinc oxide and 2 mg of cupric oxide.

In some embodiments, the second active agent is selected from the group consisting of: an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, or a carotenoid.

In some embodiments, the second active agent is mometasone furoate, tacrolimus, quercetin, or diphenhydramine.

In some embodiments, the second active agent is a flavonoid, a coumarin, a phenol or a terpenoid. In some embodiments the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), 0-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the second active agent is: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, or combinations thereof.

Specific, non-limiting examples of possible combination therapies include use of elamipretide in combination with nitric oxide (NO) inducers, statins, negatively charged phospholipids, antioxidants, minerals, anti-inflammatory agents, anti-angiogenic agents, matrix metalloproteinase inhibitors, and carotenoids. In several instances, suitable combination agents may fall within multiple categories (by way of example only, lutein is an antioxidant and a carotenoid). Further, elamipretide may also be administered with additional agents that may provide benefit to the patient, including by way of example only cyclosporin A.

In addition, elamipretide may also be used in combination with procedures that may provide additional or synergistic benefit to the patient, including, by way of example only, the use of extracorporeal rheopheresis (also known as membrane differential filtration), the use of implantable miniature telescopes, laser photocoagulation of drusen, and microstimulation therapy.

The use of antioxidants has been shown to benefit patients with macular degenerations and dystrophies. See, e.g., Arch. Ophthalmol., 119: 1417-36 (2001); Sparrow, et al., J. Biol. Chem., 278:18207-13 (2003). Examples of suitable antioxidants that could be used in combination with elamipretide include vitamin C, vitamin E, beta-carotene and other carotenoids, coenzyme Q, 4-hydroxy-2,2,6,6-tetramethylpiperidine-N-oxyl (also known as Tempol), lutein, butylated hydroxytoluene, resveratrol, a trolox analogue (PNU-83836-E), and bilberry extract.

The use of certain minerals has also been shown to benefit patients with macular degenerations and dystrophies. See, e.g., Arch. Ophthalmol., 119: 1417-36 (2001). Examples of suitable minerals that could be used in combination with elamipretide include copper-containing minerals, such as cupric oxide; zinc-containing minerals, such as zinc oxide; and selenium-containing compounds.

The use of certain negatively-charged phospholipids has also been shown to benefit patients with macular degenerations and dystrophies. See, e.g., Shaban & Richter, Biol. Chem., 383:537-45 (2002); Shaban, et al., Exp. Eye Res., 75:99-108 (2002). Examples of suitable negatively charged phospholipids that could be used in combination with elamipretide include cardiolipin and phosphatidylglycerol. Positively-charged and/or neutral phospholipids may also provide benefit for patients with macular degenerations and dystrophies when used in combination with elamipretide.

The use of certain carotenoids has been correlated with the maintenance of photoprotection necessary in photoreceptor cells. Carotenoids are naturally-occurring yellow to red pigments of the terpenoid group that can be found in plants, algae, bacteria, and certain animals, such as birds and shellfish. Carotenoids are a large class of molecules in which more than 600 naturally occurring carotenoids have been identified. Carotenoids include hydrocarbons (carotenes) and their oxygenated, alcoholic derivatives (xanthophylls). They include actinioerythrol, astaxanthin, canthaxanthin, capsanthin, capsorubin, β-8′-apo-carotenal (apo-carotenal), β-12′-apo-carotenal, α-carotene, β-carotene, “carotene” (a mixture of α- and β-carotenes), γ-carotenes, β-cyrptoxanthin, lutein, lycopene, violerythrin, zeaxanthin, and esters of hydroxyl- or carboxyl-containing members thereof. Many of the carotenoids occur in nature as cis- and trans-isomeric forms, while synthetic compounds are frequently racemic mixtures.

In humans, the retina selectively accumulates mainly two carotenoids: zeaxanthin and lutein. These two carotenoids are thought to aid in protecting the retina because they are powerful antioxidants and absorb blue light. Studies with quails establish that groups raised on carotenoid-deficient diets had retinas with low concentrations of zeaxanthin and suffered severe light damage, as evidenced by a remarkably high number of apoptotic photoreceptor cells, while the group with high zeaxanthin concentrations had minimal damage. Examples of suitable carotenoids for in combination with elamipretide include lutein and zeaxanthin, as well as any of the aforementioned carotenoids.

Suitable nitric oxide inducers include compounds that stimulate endogenous NO or elevate levels of endogenous epithelium-derived relaxing factor (EDRF) in vivo or are substrates for nitric oxide synthase. Such compounds include, for example, L-arginine, L-homoarginine, and N-hydroxy-L-arginine, including their nitrosated and nitrosylated analogs (e.g., nitrosated L-arginine, nitrosylated L-arginine, nitrosated N-hydroxy-L-arginine, nitrosylated N-hydroxy-L-arginine, nitrosated L-homoarginine and nitrosylated L-homoarginine), precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid) and the substrates for nitric oxide synthase, cytokines, adenosine, bradykinin, calreticulin, bisacodyl, and phenolphthalein. EDRF is a vascular relaxing factor secreted by the epithelium and has been identified as nitric oxide or a closely related derivative thereof (Palmer et al, Nature, 327:524-526 (1987); Ignarro et al, Proc. Natl. Acad. Sci. USA, 84:9265-9269 (1987)).

Statins serve as lipid-lowering agents and/or suitable nitric oxide inducers. In addition, a relationship has been demonstrated between statin use and delayed onset or development of macular degeneration. G. McGwin, et al., British Journal of Ophthalmology, 87:1121-25 (2003). Statins can thus provide benefit to a patient suffering from an ophthalmic condition such as the macular degenerations and dystrophies, and the retinal dystrophies when administered in combination with elamipretide. Suitable statins include, by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin.

Suitable anti-inflammatory agents with which elamipretide may be used include, by way of example only, aspirin and other salicylates, cromolyn, nedocromil, theophylline, zileuton, zafirlukast, montelukast, pranlukast, indomethacin, and lipoxygenase inhibitors; non-steroidal antiinflammatory drugs (NSAIDs) (such as ibuprofen and naproxin); prednisone, dexamethasone, cyclooxygenase inhibitors (i.e., COX-1 and/or COX-2 inhibitors such as Naproxen™, or Celebrex™); statins (by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin); and disassociated steroids.

Suitable matrix metalloproteinases (MMPs) inhibitors may also be administered in combination with elamipretide in order to treat ophthalmic conditions or symptoms associated with macular or retinal degenerations. MMPs are known to hydrolyze most components of the extracellular matrix. These proteinases play a central role in many biological processes such as normal tissue remodeling, embryogenesis, wound healing and angiogenesis. However, excessive expression of MMP has been observed in many disease states, including macular degeneration. Many MMPs have been identified, most of which are multidomain zinc endopeptidases. A number of metalloproteinase inhibitors are known (see for example the review of MMP inhibitors by Whittaker M. et al, Chemical Reviews 99(9):2735-2776 (1999)). Representative examples of MMP Inhibitors include Tissue Inhibitors of Metalloproteinases (TIMPs) (e.g., TIMP-1, TIMP-2, TIMP-3, or TIMP-4), α-2-macroglobulin, tetracyclines (e.g., tetracycline, minocycline, and doxycycline), hydroxamates (e.g., BATIMASTAT, MARIMISTAT and TROCADE), chelators (e.g., EDTA, cysteine, acetylcysteine, D-penicillamine, and gold salts), synthetic MMP fragments, succinyl mercaptopurines, phosphonamidates, and hydroxaminic acids. Examples of MMP inhibitors that may be used in combination with elamipretide include, by way of example only, any of the aforementioned inhibitors.

The use of antiangiogenic or anti-VEGF drugs has also been shown to provide benefit for patients with macular degenerations and dystrophies. Examples of suitable antiangiogenic or anti-VEGF drugs that could be used in combination with elamipretide include Rhufab V2 (Lucentis™), Tryptophanyl-tRNA synthetase (TrpRS), Eye001 (Anti-VEGF Pegylated Aptamer), squalamine, Retaane™ 15 mg (anecortave acetate for depot suspension; Alcon, Inc.), Combretastatin A4 Prodrug (CA4P), Macugen™, Mifeprex™ (mifepristone—ru486), subtenon triamcinolone acetonide, intravitreal crystalline triamcinolone acetonide, Prinomastat (AG3340—synthetic matrix metalloproteinase inhibitor, Pfizer), fluocinolone acetonide (including fluocinolone intraocular implant, Bausch & Lomb/Control Delivery Systems), VEGFR inhibitors (Sugen), and VEGF-Trap (Regeneron/Aventis).

Other pharmaceutical therapies that have been used to relieve visual impairment can be used in combination with elamipretide. Such treatments include but are not limited to agents such as Visudyne™ with use of a non-thermal laser, PKC 412, Endovion (NeuroSearch A/S), neurotrophic factors, including by way of example Glial Derived Neurotrophic Factor and Ciliary Neurotrophic Factor, diatazem, dorzolamide, Phototrop, 9-cis-retinal, eye medication (including Echo Therapy) including phospholine iodide or echothiophate or carbonic anhydrase inhibitors, AE-941 (AEterna Laboratories, Inc.), Sirna-027 (Sirna Therapeutics, Inc.), pegaptanib (NeXstar Pharmaceuticals/Gilead Sciences), neurotrophins (including, by way of example only, NT-4/5, Genentech), Cand5 (Acuity Pharmaceuticals), ranibizumab (Genentech), INS-37217 (Inspire Pharmaceuticals), integrin antagonists (including those from Jerini AG and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/Albany, Mera Pharmaceuticals), D-9120 (Celltech Group plc), ATX-S10 (Hamamatsu Photonics), TGF-beta 2 (Genzyme/Celtrix), tyrosine kinase inhibitors (Allergan, SUGEN, Pfizer), NX-278-L (NeXstar Pharmaceuticals/Gilead Sciences), Opt-24 (OPTIS France SA), retinal cell ganglion neuroprotectants (Cogent Neurosciences), N-nitropyrazole derivatives (Texas A&M University System), KP-102 (Krenitsky Pharmaceuticals), and cyclosporin A.

In any case, the multiple therapeutic agents may be administered in any order or even simultaneously. If simultaneously, the multiple therapeutic agents may be provided in a single, unified form, or in multiple forms (by way of example only, either as a single solution or as two separate solutions). One of the therapeutic agents may be given in multiple doses, or both may be given as multiple doses. If not simultaneous, the timing between the multiple doses may vary from more than zero weeks to less than about four weeks, less than about six weeks, less than about 2 months, less than about 4 months, less than about 6 months, or less than about one year. In addition, the combination methods, compositions and formulations are not to be limited to the use of only two agents. By way of example only, elamipretide may be provided with at least one antioxidant and at least one negatively charged phospholipid; or elamipretide may be provided with at least one antioxidant and at least one inducer of nitric oxide production; or elamipretide may be provided with at least one inducer of nitric oxide productions and at least one negatively charged phospholipid; and so forth.

In addition, elamipretide may also be used in combination with procedures that may provide additional or synergistic benefits to the patient. Procedures known, proposed or considered to relieve visual impairment include but are not limited to “limited retinal translocation”, photodynamic therapy (including, by way of example only, receptor-targeted PDT, Bristol-Myers Squibb, Co.; porfimer sodium for injection with PDT; verteporfin, QLT Inc.; rostaporfin with PDT, Miravent Medical Technologies; talaporfin sodium with PDT, Nippon Petroleum; motexafin lutetium, Pharmacyclics, Inc.), antisense oligonucleotides (including, by way of example, products tested by Novagali Pharma SA and ISIS-13650, Isis Pharmaceuticals), laser photocoagulation, drusen lasering, macular hole surgery, macular translocation surgery, implantable miniature telescopes, Phi-Motion Angiography (also known as Micro-Laser Therapy and Feeder Vessel Treatment), Proton Beam Therapy, microstimulation therapy, Retinal Detachment and Vitreous Surgery, Scleral Buckle, Submacular Surgery, Transpupillary Thermotherapy, Photosystem I therapy, use of RNA interference (RNAi), extracorporeal rheopheresis (also known as membrane differential filtration and Rheotherapy), microchip implantation, stem cell therapy, gene replacement therapy, ribozyme gene therapy (including gene therapy for hypoxia response element, Oxford Biomedica; Lentipak, Genetix; PDEF gene therapy, GenVec), photoreceptor/retinal cells transplantation (including transplantable retinal epithelial cells, Diacrin, Inc.; retinal cell transplant, Cell Genesys, Inc.), and acupuncture.

Further combinations that may be used to benefit an individual include using genetic testing to determine whether that individual is a carrier of a mutant gene that is known to be correlated with AMD. By way of example only, defects in the human ABCA4 gene are thought to be associated with five distinct retinal phenotypes including Stargardt disease, cone-rod dystrophy, age-related macular degeneration, and retinitis pigmentosa. See e.g., Allikmets et al., Science, 277:1805-07 (1997); Lewis et al., Am. J Hum. Genet., 64:422-34 (1999); Stone et al., Nature Genetics, 20:328-29 (1998); Allikmets, Am. J Hum. Gen., 67:793-799 (2000); Klevering, et al., Ophthalmology, 11 1:546-553 (2004). Patients possessing such a mutation are expected to find therapeutic and/or prophylactic benefit in the methods described herein.

In some embodiments, practice of the methods disclosed herein combines administration of elamipretide in combination with the administration of one or more therapies targeting the complement system. For example, the disclosure contemplates concomitant administration of a complement inhibitor such as pegcetacoplan (a.k.a., Syfovre™), avacincaptad pegol (a.k.a., Izervay™), ANX007, or a combination of pozelimab and cemdisiran, or pharmaceutically acceptable salts thereof. Individually, Pegcetacoplan (a.k.a., Syfovre™) and avacincaptad pegol (a.k.a., Izervay™) have been approved by the FDA for the treatment of geographic atrophy. One is a complement 3 inhibitor and the other is a complement 5 inhibitor. In some embodiments, multiple therapeutic agents (i.e., elamipretide, pegcetacoplan, avacincaptad pegol, ANX007 and/or pozelimab in combination with cemdisiran) may be administered in any order or even simultaneously. Typically, the therapeutic agents will be administered separately. Elamipretide is generally administered by daily subcutaneous injection while pegcetacoplan can be generally administered by intravitreal injection once every 25 to 60 days and avacincaptad pegol can be administered by intravitreal injection one every once every 21 to 35 days. In its most recent clinical trial, ANX007 was administered in two intravitreal (IVT) injections separated by 4 weeks. In a recent clinical trial, the combination of pozelimab and cemdisiran was administered subcutaneously.

As used herein, “ANX007” refers to a complement Clq inhibitor monoclonal antibody antigen-binding fragment (Fab) that binds to Clq; Clq being a molecule that binds to photoreceptor synapses to active the complement pathway, leading to inflammation and cell (photoreceptor) loss.

As used herein, the term “avacincaptad pegol” (a.k.a., Izervay™) refers to a complement-inhibiting aptamer having the structure shown below:

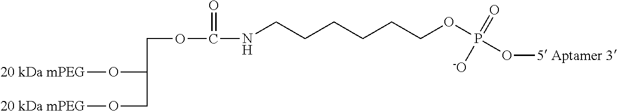

wherein the aptamer sequence is fCmGfCfCGfCmGmGfUfCfUfCmAmGmGfCGfCfUmGmAmGfUfC fUmGmAmGfUfUfUAfCfCfUmGfCmG-3T, wherein fC and fU are 2′ fluoro nucleotides, mG and mA are 2′-OMe nucleotides, all other nucleotides are 2′-OH, and 3T indicates an inverted deoxythymidine. The term “avacincaptad pegol” also refers to a complement-inhibiting aptamer having the structure shown below, wherein n=approximately 485:

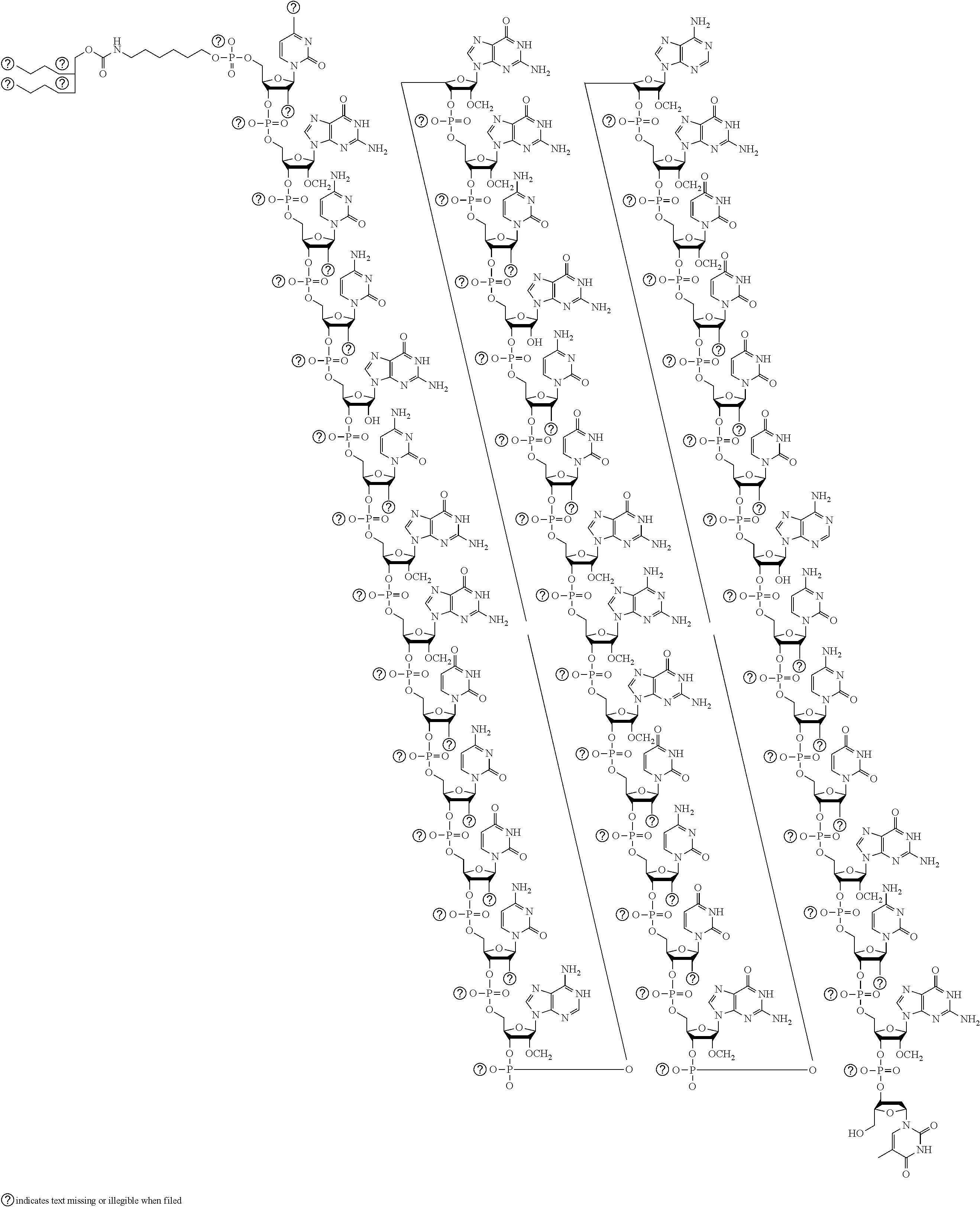

As used herein “cemdisiran” refers to a molecule comprising N-acetylgalactosamine (GalNAc) conjugated to a small-interfering RNA (siRNA) therapeutic, currently under development for the treatment of complement-mediated disease by suppressing production of complement 5 (C5) protein.

As used herein, the term “pegcetacoplan” (a.k.a., Syfovre™) refers to a complement inhibitor with the chemical formula C₁₉₇₀H₃₈₄₈N₅₀O₉₄₇S₄ and having the structure shown below:

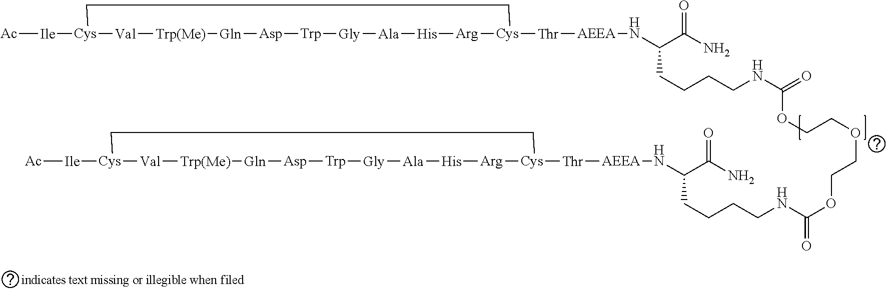

As used herein “Pozelimab” (a.k.a., REGN3918) refers to a fully human IgG4 anti-C5 monoclonal antibody that binds to C5 protein and C5 variants thereby blocking C5 cleavage into pro-inflammatory components and preventing the complement-mediated destruction of cells.

### EXAMPLES

The present disclosure is further illustrated by the following examples, which should not be construed as limiting in any way.

### Example 1—Evaluation of Elamipretide in Age-Related Macular Degeneration (AMD) in Human Subjects

This example demonstrates the efficacy of elamipretide in the treatment of human subjects having dry age-related macular degeneration (AMD) with non-central geographic atrophy (GA) in at least one eye.

### Patients/Materials and Methods

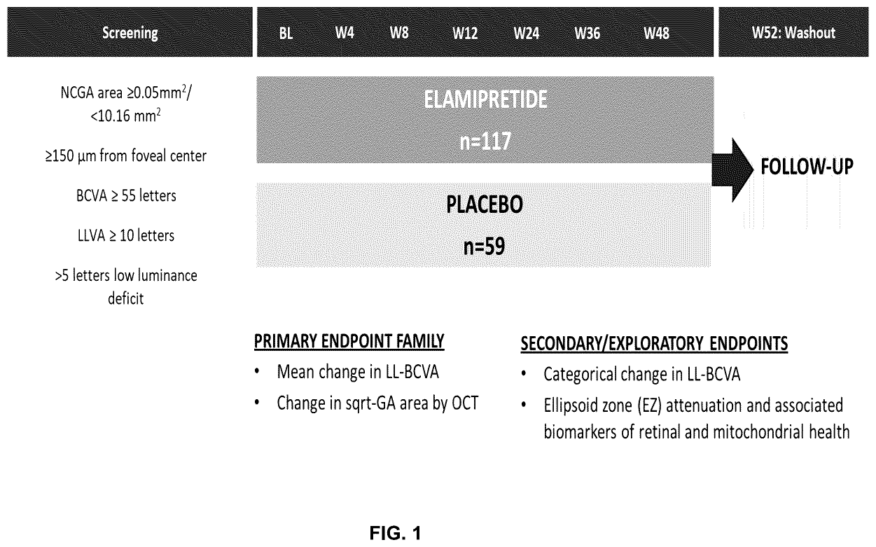

Study design. In this Phase 2, randomized, placebo-controlled, double-masked, multicenter, safety and efficacy trial (ClinicalTrials.gov identifier NCT03891875), patients with at least one eye with dry AMD and GA were included. Visit 1 (screening visit) was performed no more than 14 days before the baseline visit (visit 2, day 1) (FIG. 1). Eligible patients returned for the baseline visit, at which time they were randomized (interactive response system) in a 2:1 ratio to receive either elamipretide 40 mg or placebo administered with a pen injector delivery system as a single daily self-administered subcutaneous injection. This dose was chosen based on the systemic exposure and safety profiles of elamipretide in an earlier trial (Allingham 2022; Mettu 2022). Patients were treated for 48 weeks, with assessments at day 1 and weeks 4, 8, 12, 24, 36, and 48 (visits 2 to 8) plus a final safety follow-up visit 4 weeks after the last day of study treatment (week 52; visit 9). Patient diaries and vial counts were used and reviewed to verify compliance.

The study was conducted in accordance with consensus ethics principles derived from international ethics guidelines including the Council for International Organizations of Medical Sciences International Ethical Guidelines and the International Conference on Harmonization Good Clinical Practice Guideline. The study was approved by the study site's institutional review board and all patients provided written informed consent.

Patients—Key inclusion criteria. The original protocol specified that adults≥55 years of age with one eye with dry AMD and GA were eligible for study entry. However, a subsequent amendment to the protocol limited inclusion to patients with noncentral GA. Noncentral GA and area was determined primarily by fundus autofluorescence (FAF) and all noncentral GA lesions were required to be ≥150 μm from foveal center with preserved outer retinal structural details, as confirmed by the central reading center (BIRC, Boston MA). Noncentral GA was required to be ≥0.05 mm² and ≤10.16 mm² in size, and reside completely within the FAF 30- or 35-degree image. In the study eye, patients could not have evidence of MNV by history, optical coherence tomography (OCT), or fluorescein angiography, had a best-corrected visual acuity (BCVA) by ETDRS score of ≥55 letters, a low-luminance (LL) BCVA by ETDRS score of ≥10 letters, or an LL-BCVA deficit (defined as the difference between BCVA and LL BCVA) of >5 letters. The fellow eye was allowed to have AMD with or without GA, MNV, central GA, or no pathology.

Patients—Key exclusion criteria. Key exclusion criteria for study eye included absence of observable hyper-FAF at the margins of the GA (only for lesions 0.25 mm²), atrophic retinal disease of causality other than AMD including myopia-related maculopathy and monogenetic macular dystrophies including pattern dystrophy and adult-onset Stargardt disease. Presence or diagnosis of exudative AMD or MNV, presence of retinal vein occlusion or vitreous hemorrhage, history of retinal detachment or macular hole, presence of an epiretinal membrane that causes distortion of the retinal contour, vitreomacular traction, or advanced glaucoma in the study eye were also not allowed. Full inclusion/exclusion criteria are summarized in Table A.

TABLE A
Ocular Inclusion and Exclusion Criteria

Inclusion Criteria
Study Eye
Non-central GA was defined as well-demarcated area(s) of GA (presence and will
be determined primarily by FAF); all GA lesions must be ≥150 μm from foveal
center with preserved outer retinal structural details, as confirmed by the reading
center
GA may be multifocal, but the cumulative GA lesion size must be ≥0.05 mm² and
<10.16 mm² in size and reside completely within the FAF imaging field (FAF 30 or
35 degree image)
No evidence of MNV by history, OCT or FA in the study eye
BCVA by ETDRS score of ≥55 letters (Snellen equivalent ≥20/70) in the study eye
at the screening visit and baseline visit
LL BCVA by ETDRS score of ≥10 letters in the study eye at the screening visit and
baseline visit
LL-BCVA deficit (defined as difference the between BCVA and LL BCVA) of >5
letters in the study eye at screening and baseline visit
The fellow eye may have any of the following: no AMD, AMD without GA (i.e.,
high-risk drusen), AMD with GA, MNV AMD, or central GA. Ongoing treatment
with antiangiogenic therapies in the fellow eye was allowable
Sufficiently clear ocular media, adequate pupillary dilation, fixation to permit
quality fundus imaging, and ability to cooperate sufficiently for adequate
ophthalmic visual function testing and anatomic assessment in the study eye
Exclusion Criteria
Study Eye
Atrophic retinal disease of causality other than AMD including myopia-related
maculopathy and monogenetic macular dystrophies including pattern dystrophy and
adult-onset Stargardt disease
Presence or diagnosis of exudative AMD or MNV
Presence of retinal vein occlusion
Presence of diabetic retinopathy (a history of diabetes mellitus without retinopathy
is not a criterion for exclusion) in either eye
Presence of vitreous hemorrhage
History of retinal detachment
History of macular hole (stages 2 to 4)
Presence of an epiretinal membrane that causes distortion of the retinal contour
Presence of vitreomacular traction
At the screening visit, advanced glaucoma resulting in a cup to disc ratio of >0.8
History of glaucoma filtration surgery or uncontrolled glaucoma defined as
intraocular pressure >22 mmHg at baseline despite anti-glaucoma treatment with or
without topical antihypertensive eye drops in the study eye OR currently using >2
medications (note: combination medications count as 2 medications)
Presence of visually significant cataract OR presence of significant posterior
capsular opacity in the setting of pseudophakia. Significant cataract is defined as >+2
nuclear sclerosis or any posterior subcapsular cataract
Presence of significant keratopathy or any other media or corneal opacity that
would cause scattering of light or alter visual function, especially in low-luminance
conditions
Ocular incisional or laser surgery (including cataract surgery) within 90 days before
day 1
Yag laser capsulotomy in the study eye within 30 days before day 1
Aphakia
History of vitrectomy surgery, submacular surgery, or any vitreoretinal surgery
Prior treatment with Visudyne ® (verteporfin) ocular photodynamic therapy, external
beam radiation therapy (for intraocular conditions), or transpupillary thermotherapy
History of subthreshold laser treatment or other forms of photobiomodulation for
AMD
Intravitreal drug delivery in the past 60 days or 5 half-lives of the injected drug
whichever is longer (e.g., intravitreal corticosteroid injection, anti-angiogenic
drugs, or device implantation)
Current use of medications known to be toxic to the lens, retina, or optic nerve
(e.g., deferoxamine, chloroquine/hydroxychloroquine [Plaquenil ®], tamoxifen,
phenothiazines, ethambutol, digoxin, and aminoglycosides) from the screening visit
through the completion of the trial
Concurrent disease in either the study eye or fellow control eye that could require
medical or surgical intervention during the study period
Either Eye
History of herpetic infection
Active uveitis and/or vitreous (grade trace or above)
History of idiopathic or autoimmune-associated uveitis
Active infectious conjunctivitis, keratitis, scleritis, or endophthalmitis
BCVA = best-corrected visual acuity;
MNV = choroidal neovascularization;
ETDRS = Early Treatment Diabetic Retinopathy Study;
FA = fluorescein angiography;
FAF = fundus autofluorescence;
GA = geographic atrophy;
LL = low-luminance;
OCT = optical coherence tomography.

Absence of observable hyper-FAF at the margins of the GA (only for lesions≥0.25 mm²)

Study objectives and endpoints. The primary efficacy endpoints were changes from baseline in LL BCVA and in square root converted GA area as measured by OCT. Secondary efficacy endpoints included changes in ellipsoid zone integrity, categorical changes from baseline in LL-BCVA, changes from baseline in LL reading acuity (LLRA), BCVA, and GA area as measured by FAF. Additional predefined endpoints included change in center 1-mm and 2-mm mean EZ-RPE thickness, National Eye Institute Visual Function Questionnaire (VFQ-39) score, RA at standard light, visual function by the Low-Luminance Questionnaire (LLQ). For efficacy endpoints, the unit of analysis was study eye; in cases where both eyes are eligible for analysis, the study eye was the eye with the worse LL BCVA as determined at baseline (right eye was the study eye for equal LL BCVA). Ellipsoid zone integrity was measured as previously described on OCT utilizing a machine learning enhanced multi-layer segmentation platform that provided panmacular assessment of the EZ and RPE location (Sirici, et al., Ophthamol Surg Lasers Imaging Retina, 2022; Itoh, et al, Br J Ophthamol, 2016).

The primary safety objective was to evaluate the safety/tolerability of elamipretide. Safety/tolerability evaluations included assessment of the incidence and severity of adverse events/adverse device effects, incidence of conversion to MNV, changes from baseline in vital sign measurements, electrocardiograms, clinical evaluations, clinical laboratory evaluations, slit lamp examination, and dilated fundus examination.

Statistical analysis. Safety and efficacy variables were summarized descriptively. The safety population included all patients receiving ≥1 dose of investigational product. Efficacy was assessed in the modified ITT population (mITT), which included all randomized patients receiving ≥1 dose of study treatment and have ≥1 post-baseline value for LL BCVA or GA area on OCT. Patients were analyzed according to the actual treatment received. A sample size of 180 patients provides ≥80% power to detect a 5-letter (1-line) change from baseline mean difference in LL BCVA between drug and placebo, assuming a standard deviation of 11 letters, at a two-sided alpha-level of 0.10, and proves approximately 80% power to detect a 30% difference in the change from baseline in square root transformed total GA area by OCT between drug and placebo, assuming a standard deviation of 0.2 mm/year, and an average change of 0.33 mm/year, at a two-sided alpha-level of 0.1.

For continuous data, a mixed model for repeated measures was used for each eye separately (study eye), with fixed effects for treatment arm, study visit, the treatment arm-by-visit interaction, baseline as a covariate, a baseline-by-visit interaction, a random effect for subject, using an unstructured covariance structure. The primary time point for analysis was week 48 (end of treatment visit) and the mITT population was the primary analysis population using observed data. A family-wise alpha level of 0.1 was maintained for the primary endpoint family, using Hochberg's procedure at the primary time point of 48 weeks. If both primary endpoints were significantly different from placebo at the 0.1 (two-sided) level of significance (in favor of treatment), then both were considered statistically significant. Otherwise, the endpoint with the smaller p-value of the two was considered statistically significant, if statistically significant at the 0.05 (two-sided) level of significance.

### Results

Patients. There were 176 patients randomized in the study (elamipretide n=117; placebo n=59). Of these, 114 and 59 patients, respectively, were included in the mITT populations. Forty-two patients (elamipretide n=34 [29%]; placebo n=8 [14%]) discontinued the study early. The most common reasons for early discontinuation were withdrawal by subject (elamipretide n=20; placebo n=3) and adverse events (elamipretide n=10; placebo=4) (Table 1). The mean age was 76 years in both treatment arms and the mean (SD) baseline BCVA values were 75.8 (9.09) and 76.6 (7.90), while LL BCVA values were 53.4 (16.17) and 58.8 (10.70) for the elamipretide and placebo groups, respectively. Twenty-seven patients (elamipretide n=18; placebo n=9) with central GA were included prior to a protocol amendment to only include patients with noncentral GA. Baseline ocular parameters were generally similar between treatment groups, although patients in the elamipretide group tended to have greater disease burden at baseline as reflected by greater LL deficit (defined below), tEZa, and pEZa and lower center 1 mm mean EZ-RPE thickness compared with the placebo group (Table 2).

TABLE 1
Reasons for discontinuation

| | Elamipretide | Placebo | Overall |
|---|---|---|---|
| Reason for Discontinuation | n (%) | n (%) | n (%) |
| Subjects leaving study early | 34 (29.1) | 8 (13.6) | 42 (23.9) |
| Related to Covid | 5 (4.3) | 0 | 5 (2.8) |

Reason for early discontinuation

| | | | |
|---|---|---|---|
| Adverse event | 10 (8.5) | 4 (6.8) | 14 (8.0) |
| Lost to follow-up | 1 (0.9) | 0 | 1 (0.6) |
| Physician decision | 1 (0.9) | 1 (0.7) | 2 (1.1) |
| Protocol violation | 1 (0.9) | 0 | 1 (0.6) |
| Withdrawal by subject | 20 (17.1) | 3 (5.1) | 23 (13.1) |
| Other | 1 (0.9) | 0 | 1 (0.6) |

TABLE 2
Patient disposition and baseline characteristics (mITT population)*

| | Elamipretide | Placebo |
|---|---|---|
| Characteristic | (n = 114) | (n = 58) |
| Age, years | 76.0 (8.4) | 75.8(8.8) |
| LL BCVA | 53.4 (16.17) | 58.8 (10.70) |
| BCVA | 75.8 (9.10) | 76.6 (7.90) |
| LL-Deficit | 22.4 (12.34) | 17.8 (8.07) |
| Sqrt GA area by OCT, mm | 1.47 (0.76) | 1.38 (0.68) |
| GA area by OCT, mm² | 2.73 (2.42) | 2.37 (2.17) |
| OCT GA distance to fovea | 0.49 (0.37) | 0.45 (0.35) |
| Extrafoveal/ foveal, n (%) | 96 (84)/18 (16) | 49 (84)/9 (16) |
| Multifocal/unifocal, n (%) | 85 (73)/32 (27) | 43 (74)/15 (26) |
| Percent of total EZ attenuation, | 16.01 (12.46) | 12.20 (8.75) |
| Percent of partial EZ attenuation | 25.94 (19.58) | 20.82 (15.29) |
| Center 1 mm Mean EZ-RPE | 16.97 (12.66) | 18.85 (12.11) |
| thickness | | |

*±SD unless otherwise indicated
BCVA = best corrected visual acuity;
EZ = ellipsoid zone;
GA = geographic atrophy;
mITT = modified intent-to-treat;
OCT = optical coherence tomography;
LL = low luminance;
RPE = retinal pigment epithelium;
SD = standard deviation;
Sqrt = square root

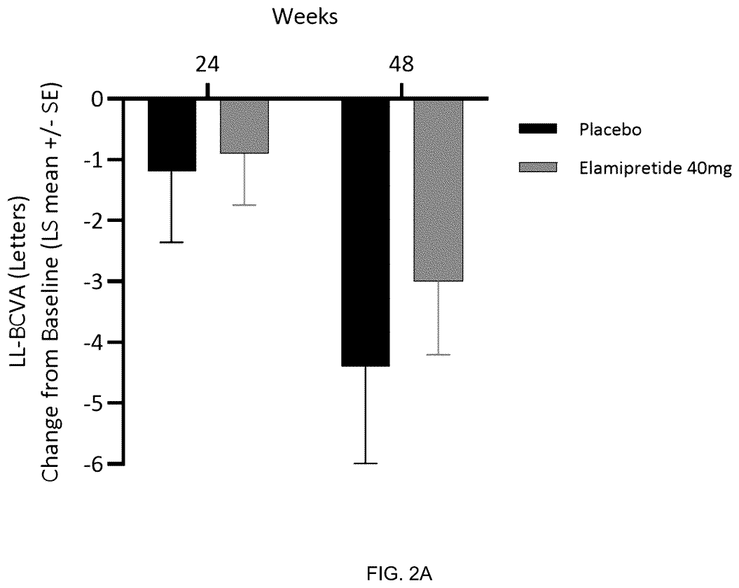

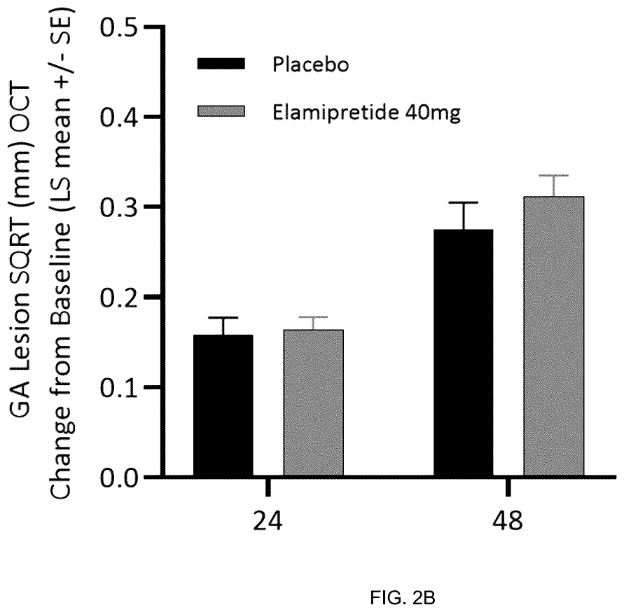

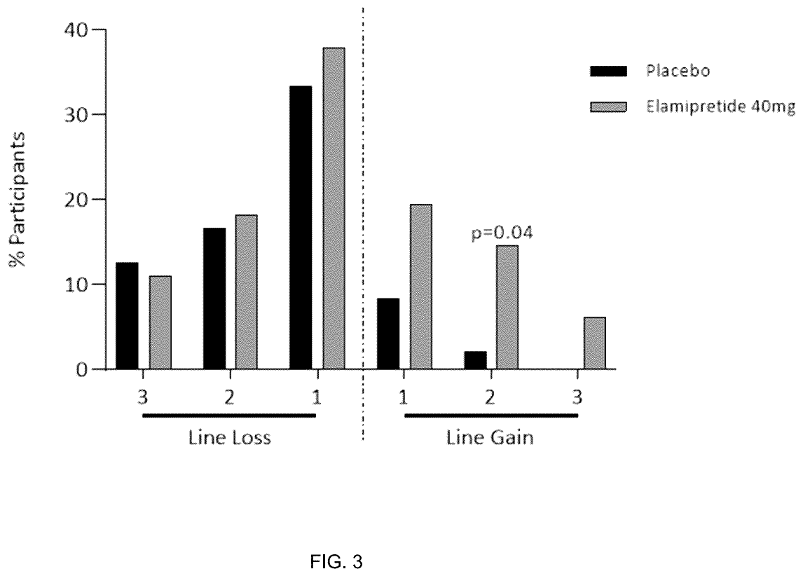

Efficacy. The primary endpoint of least squares (LS) mean changes from baseline in LL BCVA (−3.0 letters in the elamipretide group versus −4.4 letters in the placebo group; P=0.49) is shown in FIG. 2A, and the square root of GA area progression as measured by OCT (0.312 mm elamipretide versus 0.275 mm placebo; P=0.34) is shown in FIG. 2B. The elamipretide and placebo groups for the LS mean (SD) change from baseline to week 48 for the secondary endpoints of LLRA (0.024 [0.0359] vs 0.069 [0.0468], BCVA (−3.3 [0.77] vs −2.6 [1.00]; P=0.5642) and change in GA area was measured by FAF (1.099 [0.0.0761] vs 0.958 [0.1013]; P=0.2672). Elamipretide treatment was associated with significantly more patients experiencing a ≥2-line gain (≥10 letter) in LL BCVA versus placebo (14.6% vs 2.1%; P=0.0404) (FIG. 3). The percentage of patients at week 48 experiencing a >1-line gain on elamipretide was higher compared to placebo.

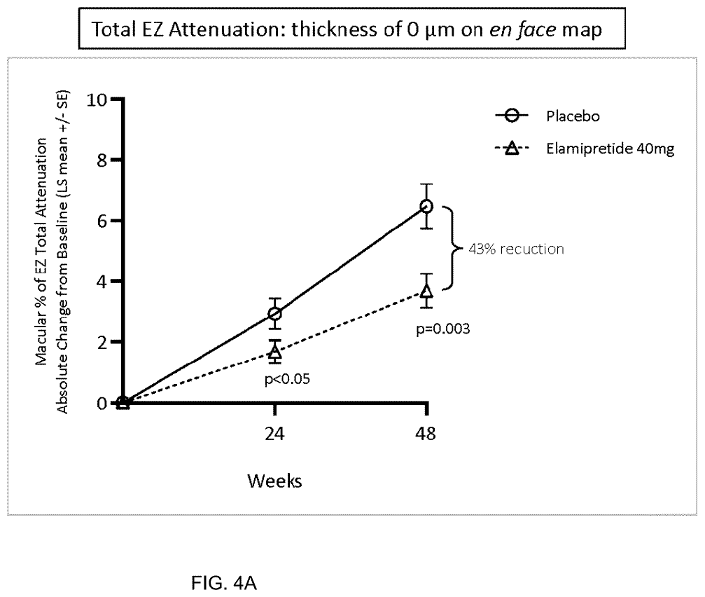

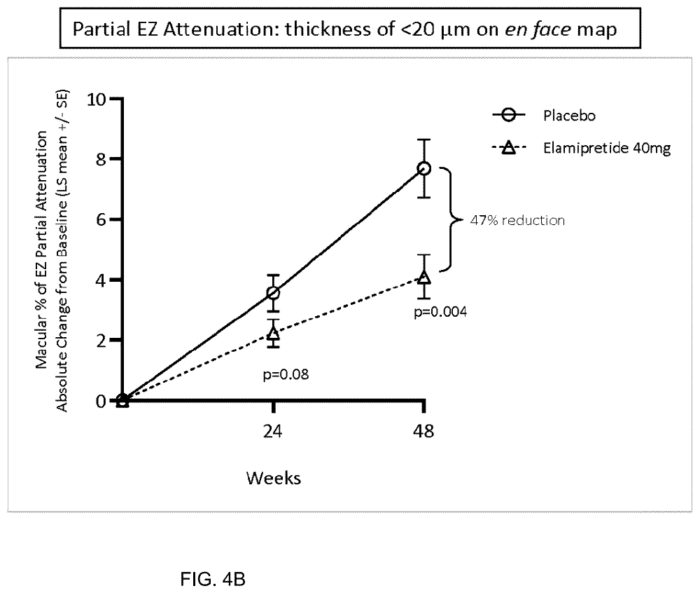

In pre-specified secondary endpoints, elamipretide was associated with significantly less progression of tEZa. Specifically, the LS mean change from baseline to week 48 for macular percentage of tEZa (i.e., EZ-RPE thickness of 0 μm) was 43% lower (3.69% [0.562] vs 6.47% [0.737]; P=0.0034) in favor of the elamipretide group (FIG. 4A). Similarly, the LS mean change from baseline to week 48 for pEZa (i.e., EZ-RPE thickness≤20 μm) was 47% lower (4.10% [0.737] vs 7.68% [0.969]; P=0.0040) in the elamipretide group versus placebo (FIG. 4B). Post-hoc analysis showed that LL BCVA changes were correlated with the change in tEZ attenuation (P<0.01). A separate post-hoc analysis identified a significant correlation between baseline center 1-mm EZ-RPE thickness and change in LL BCVA at week 48 (r=0.26, p=0.02) in the elamipretide treatment group. Table 3 shows the results of a post hoc analysis conducted by converting percent of partial EZ attenuation and percent of total EZ attenuation to total attenuation area (mm²) by multiplying the applicable percent EZ attenuation by the total macular area on the OCT scan allows for an easier comparison of EZ characteristics to GA area. The areas of percent of partial EZ attenuation and percent of total EZ attenuation were much larger than GA at baseline in both treatment groups (Table 3). The mITT population was used for the analysis. Partial EZ attenuation and total EZ attenuation, elamipretide n=72 and placebo n=41, at 48 weeks. Geographic atrophy: elamipretide n=76 and placebo n=45, at 48 weeks. Statistical analysis showing nominal “p values.”

TABLE 3
Zonal Lesion Area Growth (mm²): partial EZ attenuation (pEZa),
total EZ attenuation (tEZa), and geographic atrophy (GA)

| | | Change from | | | Change from |
|---|---|---|---|---|---|
| | | baseline at | | | baseline at |
| | Baseline | Week 48 | | Baseline | Week 48 |
| Elamipretide | (mm²) | (mm²) | Placebo | (mm²) | (mm²) |
| pEZa | 9.03 | 1.34 | pEZa | 7.75 | 2.65 |
| | | | | | p = 0.01 |
| | | | | | vs. ELAM |
| tEZa | 5.39 | 1.39 | tEZa | 4.67 | 2.23 |
| | | | | | p = 0.04 |
| | | | | | vs. ELAM |
| GA | 2.63 | 1.15 | GA | 2.50 | 0.99 |

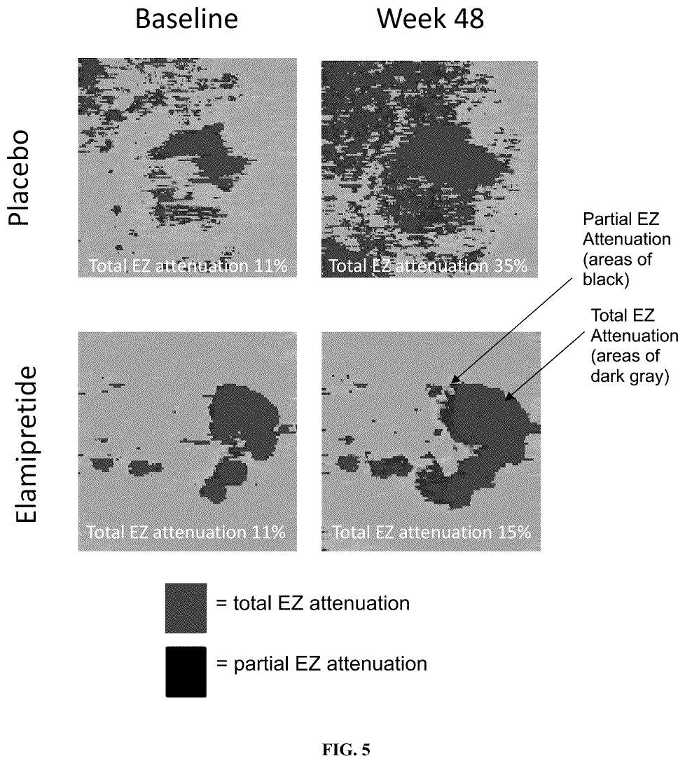

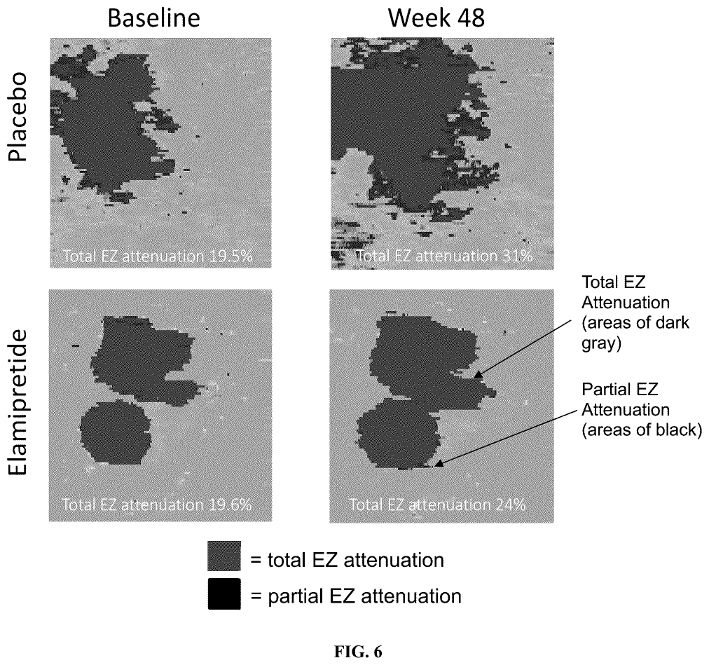

Representative examples of EZ attenuation alterations are shown in FIG. 5 and FIG. 6.

A post-hoc analysis showed that LL-BCVA changes were correlated with the change in tEZ attenuation (P<0.01). A separate post-hoc analysis identified a significant correlation between baseline center 1 mm EZ-RPE thickness and change in LL BCVA at week 48 (r=0.26, p=0.02) in the elamipretide treatment group.

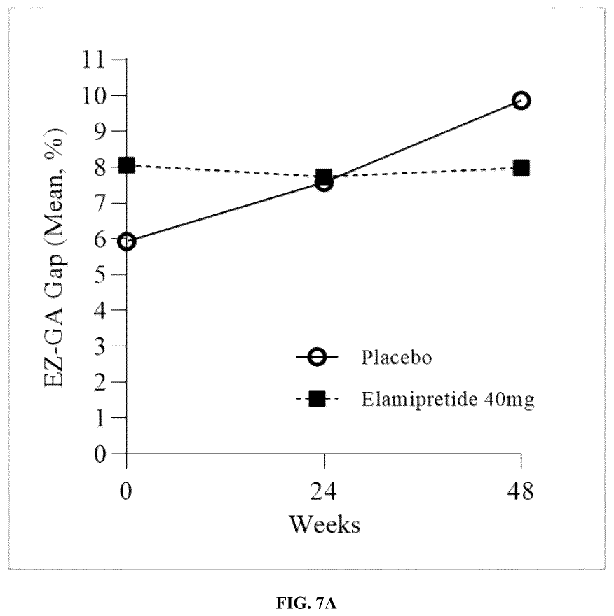

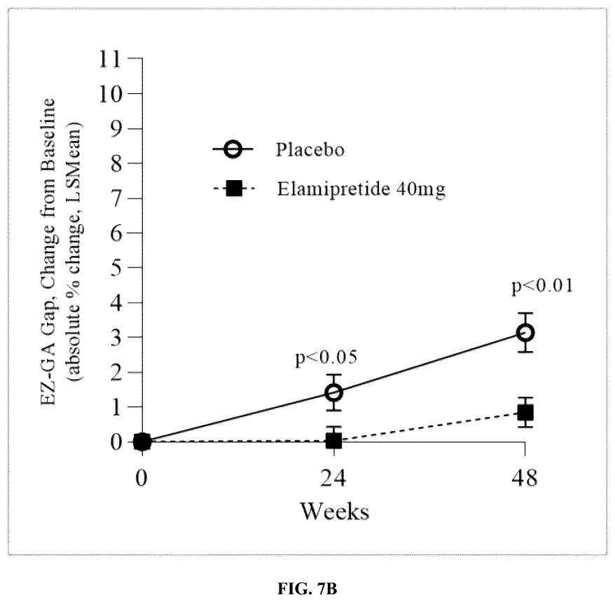

In a post-hoc analysis, treatment with elamipretide was associated with a slower rate of progression of the EZ-GA gap (FIGS. 7A-7B).

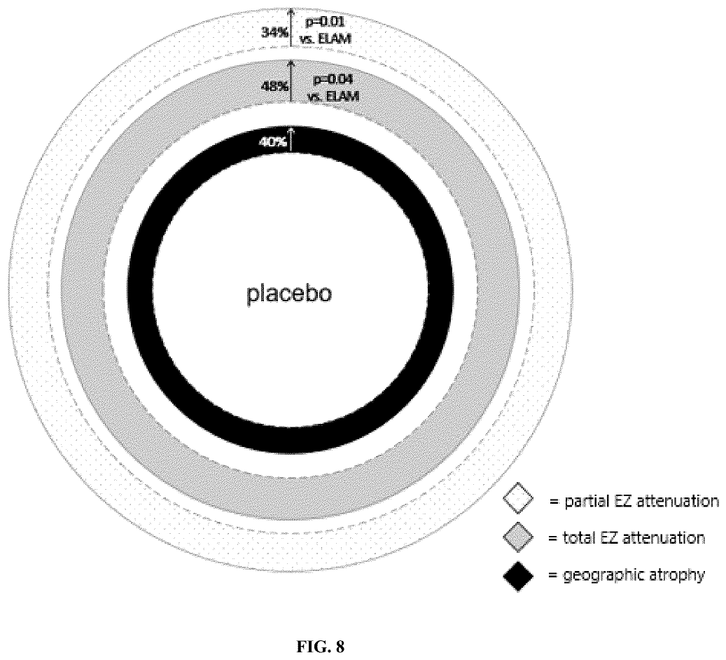

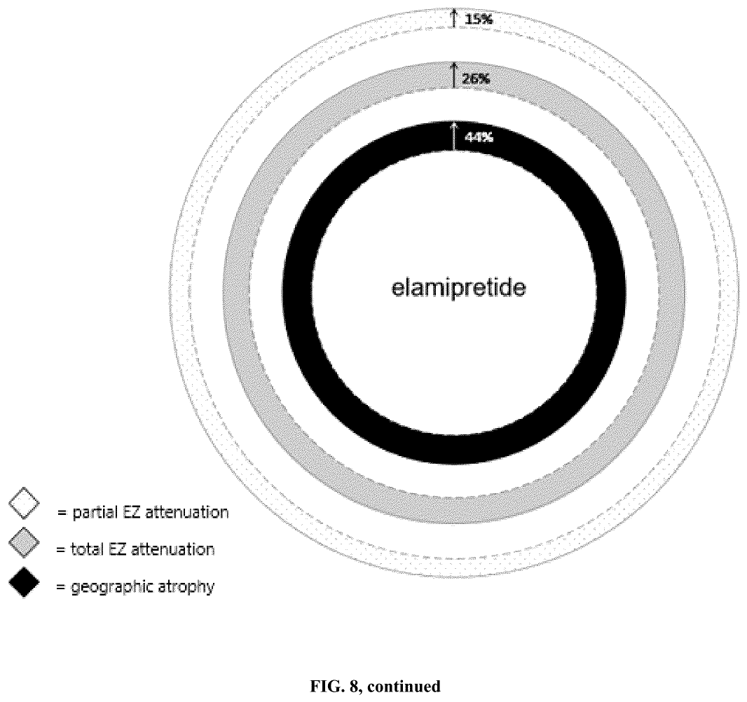

As shown in Table 3, total EZ attenuation (tEZa) was greater in the elamipretide treated arm at baseline. During the trial, progression of EZ attenuation was significantly greater in the placebo treatment arm, with total EZ attenuation area (mm²) growing almost twice as fast in the placebo arm (48%) as in the elamipretide arm (26%), and partial EZ attenuation area growing more than twice as fast in the placebo arm (34%) as in the elamipretide arm (15%). Moreover, in the placebo arm, geographic atrophy (GA) area progressed to near the baseline boundary of total EZ attenuation and partial EZ attenuation continued to expand its boundary. Conversely, in the elamipretide treated arm, despite greater total EZ attenuation at baseline, GA area did not progress to the baseline boundary of total EZ attenuation during the trial and the loss of previously normal EZ was minimized. See FIG. 8 for a graphical illustration.

Post-hoc analyses were conducted to characterize the relationship between elamipretide's effect on photoreceptor integrity (i.e., the sum of total EZ attenuation and partial EZ attenuation) and visual function (LL BCVA). This analysis demonstrated that LL BCVA change was correlated with the change in macular percentage of total EZ attenuation at week 48 (r=−0.35, p<0.0001). A second analysis showed a significant correlation between baseline center 1 mm EZ-RPE thickness and change in LL BCVA at week 48 (r=0.26, p=0.02). In this analysis, a comparison of treatment vs. placebo in the subgroup of patients with a mean center 1 mm EZ-RPE thickness≥10-micron trended towards a treatment effect in LL BCVA.

### SUMMARY

The results of this experiment demonstrate that elamipretide was associated with significantly more patients experiencing a ≥2-line gain at Week 48 in LL BCVA versus placebo. The data demonstrate that 48-weeks of elamipretide therapy resulted in significantly less progression of total EZ attenuation (a 43% reduction) and partial EZ attenuation (a 47% reduction) relative to placebo, and that elamipretide was protective of EZ attenuation. Accordingly, these results demonstrate that elamipretide is useful in methods of treating, preventing or delaying (slowing) age-related macular degeneration (AMD) as well as geographic atrophy (GA) secondary to age-related macular degeneration (AMD).

### Example 2—Treatment of Age-Related Macular Degeneration (AMD) in Human Subjects

The effects of elamipretide in treating age-related macular degeneration (AMD) are investigated in humans.

### Study Design

Subjects having or suspected of having non-exudative (dry) age-related macular degeneration (dry AMD) in at least one eye will be enrolled in the study. Subjects will be screened for baseline ocular parameters including age, low luminance best corrected visual acuity (LL BCVA), best corrected visual acuity (BCVA), LL deficit (or LLD, which is calculated by subtracting the LL-BCVA from the BCVA), the square root of GA area (mm) as measured by optical coherence tomography (OCT), the GA area (mm²) as measured by OCT, the GA distance (μm) to fovea as measured by OCT, the percent of total EZ attenuation (tEZa), the total EZ attenuation area (mm²), the percent of partial EZ attenuation (pEZa), and the center 1 mm mean ellipsoid zone-retinal pigment epithelium (EZ-RPE) thickness (μm).

Following the screening period, subjects presenting with a center 1-mm EZ-RPE thickness greater than 20 μm and a tEZa that is greater than zero and less than 30% are assigned to a first treatment group (early AMD group). Subjects presenting with a center 1-mm EZ-RPE thickness of 20 μm or less and a tEZa that is greater than or equal to 30% are assigned to a second treatment group (late AMD group). Subjects in both treatment groups will receive once daily subcutaneous (SC) elamipretide (40 mg) for at least 48 weeks.

Following the treatment period, the parameters including LL BCVA, BCVA, LL-deficit, the square root of GA area (mm) as measured by optical coherence tomography (OCT), the GA area (mm²) as measured by OCT, the GA distance (μm) to fovea as measured by OCT, the percent of total EZ attenuation (tEZa), the total EZ attenuation area (mm²), the percent of partial EZ attenuation (pEZa), and the center 1 mm mean ellipsoid zone-retinal pigment epithelium (EZ-RPE) thickness (μm) will be measured, and efficacy endpoints such as changes in the BCVA, LL BCVA, GA, pEZa, and tEZa will be calculated.

### Results

It is anticipated that elamipretide treatment in the subjects meeting the criteria of the early AMD group will exhibit an enhanced improvement in the efficacy endpoints as compared to the subjects meeting the criteria of the late AMD group.

### SUMMARY

Accordingly, these results will demonstrate that elamipretide is useful in methods for treating dry AMD in subjects selected for treatment having a center 1-mm EZ-RPE thickness that is greater than 20 μm and a tEZa that is greater than zero and less than 30%.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

Each and every publication and patent mentioned in the above specification is herein incorporated by reference in its entirety for all purposes. Various modifications and variations of the described methods and system of the present technology will be apparent to those skilled in the art without departing from the scope and spirit of the present technology. Although the present technology has been described in connection with specific embodiments, the present technology as claimed should not be unduly limited to such specific embodiments. Indeed, various modifications of the described modes for carrying out the present technology which are obvious to those skilled in the art and in fields related thereto are intended to be within the scope of the following claims.

### REFERENCES

Abraham J R, Jaffe G J, Kaiser P K, et al. Impact of Baseline Quantitative OCT Features on Response to Risuteganib for the Treatment of Dry Age-Related Macular Degeneration: The Importance of Outer Retinal Integrity. Ophthalmol Retina. 2022 November; 6(11):1019-1027.
Alam N, Mills W C, Wong A A, et al. A mitochondrial therapeutic reverses visual decline in mouse models of diabetes. Dis Models Mech. 2015; 8:701-710.
Allingham M J Mettu P S, Cousins S W. Phase 1 clinical trial of elamipretide in intermediate age-related macular degeneration and high-risk drusen. Opththamol Sci. 2022; 1:100095.
Balaratnasingam C, Inoue M, Ahn S, McCann J, Dhrami-Gavazi E, Yannuzzi L A, Freund K B. Visual Acuity Is Correlated with the Area of the Foveal Avascular Zone in Diabetic Retinopathy and Retinal Vein Occlusion. Ophthalmology. 2016 November; 123(11):2352-2367. doi: 10.1016/j.ophtha.2016.07.008.
Boyer D S, Schmidt-Erfurth U, van Lookeren Campagne M, et al. The Pathophysiology of Geographic Atrophy Secondary to Age-Related Macular Degeneration and the Complement Pathway as a Therapeutic Target. Retina. 2017; 37 (5):819-835.
Bressler N M, Bressler S B, Congdon N G, et al; Age-Related Eye Disease Study Research Group. Potential public health impact of Age-Related Eye Disease Study results: AREDS report no. 11. Arch Ophthalmol. 2003 November; 121(11):1621-4.
Cabral de Guimaraes T A, Varela M D, Georgiou M, Michaelides M. Treatments for dry age-related macular degeneration: therapeutic avenues, clinical trials and future directions. Br J Opthalmol 2022; 106:297-304.
Chakravarthy U, Bailey C C, Johnston R L, et al. Characterizing Disease Burden and Progression of Geographic Atrophy Secondary to Age-Related Macular Degeneration. Ophthalmology. 2018; 125 (6):842-849.
Chandramohan A, Stinnett S S, Petrowski J T, et al. Visual Function Measures in Early and Intermediate Age-Related Macular Degeneration. Retina. 2016 May; 36(5):1021-1031.
Chen M, Liu B, Ma J, et al. Protective effect of mitochondriatargeted peptide MTP131 against oxidative stressinduced apoptosis in RGC5 cells. Mol Med Rep. 2017; 15 (4):2179-2185.
Chen M, Liu B, Gao Q, et al. Mitochondria-targeted peptide MTP-131 alleviates mitochondrial dysfunction and oxidative damage in human trabecular meshwork cells. Invest Ophthalmol Vis Sci. 2011; 52 (10):7027-7037.
Cousins S W. Role of mitochondrial dysfunction in dry age-related macular degeneration. Retina Today. 2015 (May/June):1-5.
Cousins S W. Update: Elamipretide (MTP-131), a novel mitochondrial targeting drug, for treatment of intermediate dry AMD. Presented at: Angiogenesis, Aug. 6-11, 2017; Newport, RI.
Datta S, Cano M, Ebrahimi K, et al. The impact of oxidative stress and inflammation on RPE degeneration in non-neovascular AMD. Prog Retin Eye Res. 2017; 60:201-218.
Ehlers J P, Yordi S, Cetin H, McConville C, Bonnay M, Kalra G, Cakir Y, Sassine A, Whitmore V, Borisiak K, Cohen D, Reese J, Clark J, Zhu L, Srivastava Sk. Exploration of Machine Learning-Enhanced Compartmental Retinal Integrity Assessment for Progression Risk and Treatment Response in the GATHERI Study. Macula Society. Berlin. June 2022
Ehlers J P, Uchida A, Hu M, Figueiredo N, Kaiser P K, Heier J S, Brown D M, Boyer D S, Do D V, Gibson A, Saroj N, Srivastava S K. Higher-Order Assessment of OCT in Diabetic Macular Edema from the VISTA Study: Ellipsoid Zone Dynamics and the Retinal Fluid Index. Ophthalmol Retina. 2019 December; 3(12):1056-1066.
Ehlers J P, Zahid R, Kaiser P K, Heier J S, Brown D M, Meng X, Reese J, Le T K, Lunasco L, Hu M, Srivastava S K. Longitudinal Assessment of Ellipsoid Zone Integrity, Subretinal Hyperreflective Material, and Subretinal Pigment Epithelium Disease in Neovascular Age-Related Macular Degeneration. Ophthalmol Retina. 2021 December; 5(12):1204-1213.
Etheridge T, Dobson E T A, Wiedenmann M, Odern N, VanVeldhuisern P, Scott I U, Ip M S, Eliceiri K W, Blodi B A, Domalpally A. Ellipsoid Zone Defects in Retinal Vein Occlusion Correlates With Visual Acuity Prognosis: SCORE2 Report 14. Transl Vis Sci Technol. 2021 Mar. 1; 10(3):31. doi: 10.1167/tvst.10.3.31.
Fisher C, Ferrington D. Perspective on AMD pathbiology: A bioenergetic crisis in RPE. Invest Ophthalmol Vis Sci. 2018; 59:AMD41-AMD47.
Fleckenstein Monika, Mitchell P, Freund K B, Sadda A, Holz F G, Brittain C, Henry E C, Ferrara D. The progression of geographic atrophy secondary to age-related macular degeneration. Opththamology 2018; 125:369-390.
Fu Z, Kern T S, Hellstrom A, Smith L E H. Fatty acid oxidation and photoreceptor metabolic needs. J Lipid Res 2021; 62:100035. doi: 10.1194/jirTR120000618.
Gin T J, Wu Z, Chew S K, Guyine R H, Luu C D. Quantitative Analysis of the Ellipsoid Zone Intensity in Phenotypic Variations of Intermediate Age-Related Macular Degeneration. Invest Ophthalmol Vis Sci. 2017 Apr. 1; 58(4):2079-2086.
Guymer R I, Rosenfeld P J, Curcio C A, et al. Incomplete retinal pigment epithelial and outer atrophy (iRORA) in age-related rnacular degeneration: CAM Report 4. Opthalmology 2020; 127(3):394-409.
Handa J T, Cano M, Wang L, et al. Lipids, oxidized lipids, oxidation-specific epitopes, and Age-related Macular Degeneration. Biochim Biophys Acta Mol Cell Biol Lipids. 2017; 1862 (4):430-440.
Hara T, Zhou H P, Kitano M, Kure K, Asaoka R, Inoue T, Obata R. Quantification of residual ellipsoid zone and its correlation with visual functions in patients with cone-rod dystrophy. Eur J Ophthalmol. 2021 November; 31(6):3117-3123. doi: 10,1177/1120672121990561.
Holz F G, Strauss E C, Schmitz-Valckenberg S, et al. Geographic atrophy: clinical features and potential therapeutic approaches. Ophthalmology. 2014; 121 (5):1079-1091.
Huang J, Li X, Li M, et al. Mitochondria-targeted antioxidant peptide SS31 protects the retinas of diabetic rats. Curr Mol Med. 2013; 13 (6):935-945.
Itoh Y, Vasanji A, Ehlers J P. Volumetric ellipsoid zone mapping for enhanced visualisation of outer retinal integrity with optical coherence tomography. Br J Ophthalmol. 2016 March; 100(3):295-9.
Jaffe G J, Chakravarthy U, Freund K B, et al. Imaging Features Associated with Progression to Geographic Atrophy in Age-Related Macular Degeneration: Classification of Atrophy Meeting Report 5. Ophthalmol Retina. 2021 September; 5(9):855-867.
Kapphahn R. Elamipretide protects RPE and improves mitochondrial function in models of AMD [poster]. Presented at: American Society of Cataract and Refractive Surgery, May 5-May 9, 2017; Los Angeles, C A.
Kessler L J, Auffarth G U, Bagautdinov D, Khoramnia R. Ellipsoid Zone Integrity and Visual Acuity Changes during Diabetic Macular Edema Therapy: A Longitudinal Study. J Diabetes Res. 2021 Oct. 7; 2021:8117650. doi: 10.1155/2021/8117650. PMID: 34660813; PMCID: PMC8516551.
Künzel S H, Möller P T, Lindner M, et al. Determinants of Quality of Life in Geographic Atrophy Secondary to Age-Related Macular Degeneration Invest Ophthalmol Vis Sci. 2020 May 11; 61(5):63.
Li J, Chen X, Xiao W, et al. Mitochondria-targeted antioxidant peptide SS31 attenuates high glucose-induced injury on human retinal endothelial cells. Biochem Biophys Res Commun. 2011; 404 (1):349-356.
Liang X, Chen F, Zhou H, et al. SS31 Protects human RPE cells from oxidative damage and reduces laser-induced choroidal neovascularization. Presented at: Association for Research in Vision and Ophthalmology (ARVO) Annual Meeting. Fort Lauderdale, FL, 2010.
Mai J, Riedl S, Reiter G S, et al. Comparison of Fundus Autofluorescence Versus Optical Coherence Tomography-based Evaluation of the Therapeutic Response to Pegcetacoplan in Geographic Atrophy. Am J Ophthalmol. 2022; 244: 175-182.
Mettu P S, Allingham M J, Cousins S W. Phase 1 clinical trial of elamipretide in dry age-related macular degeneration and noncentral geographic atrophy. Opththalmol Sci. 2022; 2:100086
Min K, Smuder A J, Kwon O S, et al. Mitochondrial-targeted antioxidants protect skeletal muscle against immobilization-induced muscle atrophy. J Appl Physiol (1985). 2011; 111 (5):1459-1466.
Okawa H, Sampath A P, Laughlin S B, Fain G L. ATP consumption by mammalian rod photoreceptors in darkness and in light. Curr BioL 2008 Dec. 23; 18(24):1917-21. doi: 10.1016/j.cub.2008.10.029.
Pasricha M V, Tai V, Sleiman K, et al. Local Anatomic Precursors to New-Onset Geographic Atrophy in Age-Related Macular Degeneration as Defined on OCT. Ophthalmic Res. 2021 May; 5(5):396-408.
Pauleikhoff D, Bonelli R, Dubis A M, Gunnenann F, Rothaus K, Charbel Issa P, Heeren T F, Peto T, Clemons T E, Chew E Y, Bird A C, Sallo F B; MacTel Study Group. Progression characteristics of ellipsoid zone loss in macular telangiectasia type 2. Acta Ophthalmol. 2019 November; 97(7):e998-e1005. doi: 10.1111/aos.14110.
Pennington K L, DeAngelis M M. Epidemiology of age-related macular degeneration (AMD): associations with cardiovascular disease phenotypes and lipid factors. Eye Vis (Lond). 2016; 3:34.
Pfau M, von der Emde L, de Sisternes L, et al. Progression of Photoreceptor Degeneration in Geographic Atrophy Secondary to Age-related Macular Degeneration. JAMA Ophthalmol. 2020 Oct. 1; 138(10):1026-1034.
Pondorfer S G, Heinemann M, Wintergerst M W M, et al. Detecting vision loss in intermediate age-related macular degeneration: A comparison of visual function tests. PLoS One. 2020 Apr. 16; 15(4):e0231748.
Rein D B, Wittenborn J S, Zhang X, et al. Forecasting age-related macular degeneration through the year 2050: the potential impact of new treatments. Arch Ophthalmol. 2009; 127 (4):533-540.
Riazi-Esfahani M, Kuppermann B D, Kenney M C. The role of mitochondria in AMD: Current knowledge and future applications. J Ophthalmic Vis Res. 2017; 12 (4):424-428.
Sadda S R, Guymer R, Holz F G, et al. Consensus Definition for Atrophy Associated with Age-Related Macular Degeneration on OCT: Classification of Atrophy Report 3. Ophthalmology. 2018; 125(4):537-548.
Sarici K, Abraham J R, Sevgi D D, et al. Risk Classification for Progression to Subfoveal Geographic Atrophy in Dry Age-Related Macular Degeneration Using Machine Learning-Enabled Outer Retinal Feature Extraction. Ophthalmic Surg Lasers Imaging Retina. 2022 January; 53(1):31-39.
Scholl H P N, Boyer D, Giani A, Chong V. The Use of Neuroprotective Agents in Treating Geographic Atrophy. Ophthalmic Res. 2021; 64(6):888-902.
Siegel M P, Kruse S E, Percival J M, et al. Mitochondrial-targeted peptide rapidly improves mitochondrial energetics and skeletal muscle performance in aged mice. Aging Cell. 2013; 12 (5):763-771.
Sunness J S, Rubin G S, Broman A, et al. Low luminance visual dysfunction as a predictor of subsequent visual acuity loss from geographic atrophy in age-related macular degeneration. Ophthalmology. 2008; 115 (9):1480-1488, 1488 e1481-1482.
Szeto H H. First-in-class cardiolipin-protective compound as a therapeutic agent to restore mitochondrial bioenergetics. Br J Pharmacol. 2014; 171 (8):2029-2050.
Tan L X, Germer C J, La Cunza N, Lakkaraju A. Complement activation, lipid metabolism, and mitochondrial injury: Converging pathways in age-related macular degeneration, Redox Biol. 0.2020 October; 37:101781.
Thiele S, Wu Z, Isselmann B, Pfau M, Guymer R H, Luu C D. Natural History of the Relative Ellipsoid Zone Reflectivity in Age-Related Macular Degeneration. Ophthalmology Retina 2022.
Toprak I, Yaylali V, Yildirim C. Early deterioration in ellipsoid zone in eyes with non-neovascular age-related macular degeneration. Int Ophthalmol. 2017 August; 37(4):801-806, doi: 10.1007/s10792-016-0331-3.
Woronkowicz M, Lightman S, Tomkins-Netzer O. The prognostic value of total macular external limiting membrane and ellipsoid zone damage for clinical outcome in treatment-resistant neovascular age-related macular degeneration. Graefes Arch Clin Exp Ophthalmol. 2020 November; 258(11):2373-2378. doi: 10.1007/s00417-020-04869-4.
Vogl W D, Riedl S, Mai J, Reiter G S, Lachinov D, Bogunović H, Schmidt-Erfurth U. Predicting Topographic Disease Progression and Treatment Response of Pegcetacoplan in Geographic Atrophy Quantified by Deep Learning. Ophthalmol Retina. 2023 January; 7(1):4-13

1. A method for treating, preventing or delaying (slowing) the progression of age-related macular degeneration (AMD) in a mammalian subject in need thereof, comprising the steps of:
a) selecting subjects diagnosed as having, or suspected of having, AMD;
b) examining one or both eyes of the subject using optical coherence tomography (OCT) to produce a first OCT scan for each eye examined;
c) analyzing each OCT scan for the subject to determine:
(i) the area percent of partial EZ attenuation (pEZa);
(ii) the area percent of total EZ attenuation (tEZa); and/or
(iii) the center 1-mm ellipsoid zone-retinal pigment epithelium (EZ-RPE) thickness; and
d) administering an effective amount of elamipretide to subjects presenting with AMD in at least one eye;
(i) an area percent of partial EZ attenuation (pEZa) greater than zero and less than 100 percent;
(ii) an area percent tEZa is greater than zero and less than 100 percent; and/or
(iii) a center 1-mm EZ-RPE thickness greater than 20 μm.

2.-33. (canceled)

34. A method comprising:
a) examining one or both eyes of a mammalian subject using optical coherence tomography (OCT) to produce a 1^{st} OCT scan for each eye examined;
b) reexamining one or both eyes of the subject using optical coherence tomography (OCT) to produce a 2^{nd} OCT scan for one or both of the subject's eyes on a day that is after performing the examination according to step (a);
c) optionally repeating step (b) n-times, each n^{th} reexamination occurring on a day that is after the n^{th}−1 examination, that produced an n^{th}−1 OCT scan for one or both eyes, to thereby produce a n^{th} OCT scan for one or both eyes, where n is a whole number from 3 to 100; and
d) examining the OCT scans to determine:
(i) a rate of photoreceptor loss;
(ii) a percent of photoreceptor loss; and/or
(iii) a mean change in the macular area of photoreceptor loss;
in each eye examined for the subject that has occurred from: a) the 1^{st} OCT scan to the 2^{nd} OCT scan or to any one or more of the n^{th} OCT scans; and/or b) between two later obtained OCT scans for a particular eye examined.

35.-57. (canceled)

58. A method comprising:
a) examining one or both eyes of a mammalian subject using a visual acuity test to determine the subject's 1^{st} low luminance best corrected visual acuity (1^{st} LL BCVA), collectively and/or on an eye-by-eye basis;
b) reexamining one or both eyes of the subject using a visual acuity test, to thereby determine the subject's 2^{nd} low luminance best corrected visual acuity (2^{nd} LL BCVA), collectively and/or on an eye-by-eye basis, on a day that is after performing the examination according to step (a);
c) optionally repeating step (b) n-times, to thereby determine the subject's n^{th} low luminance best corrected visual acuity (n^{th} LL BCVA), collectively and/or on an eye-by-eye basis, each n^{th} reexamination occurring on a day that is after the n^{th}−1 examination, where n is a whole number from 3 to 100; and
d) determining whether the subject has lost or gained one or more letters and/or one or more lines in LL BCVA, collectively or on an eye-by-eye basis: a) from the 1^{st} LL BCVA determination to the 2^{nd} LL BCVA determination or any one or more of the n^{th} LL BCVA determinations; and/or b) between two later LL BCVA determinations.

59.-81. (canceled)

82. A method for improving the low luminance best corrected visual acuity (LL BCVA) by 2 or more lines, collectively or on an eye-by-eye basis, in a mammalian subject having, or suspected of having, age-related macular degeneration (AMD), comprising administering elamipretide to the subject for a period of at least 12 weeks.

83-100. (canceled)

101. A method for reducing the ratio of tEZA to RPE loss as determined by an OCT scan in a mammalian subject having, or suspected of having, age-related macular degeneration (AMD), comprising administering elamipretide to the subject for a period of at least 12 weeks.

102.-119. (canceled)

120. A method for protecting against photoreceptor loss in a mammalian subject having, or suspected of having, age-related macular degeneration (AMD), comprising administering elamipretide to the subject for a period of at least 12 weeks.

121. The method of claim 120, wherein elamipretide is administered to the subject for at least 3 months, at least 6 months, at least 9 months, at least 12 months, at least 18 months, at least 24 months or at least 48 months.

122. The method of claim 120, wherein protection against photoreceptor loss is determined by measuring the difference percentage of tEZa between two time points, each as determined by examination or an OCT scan or scans, between a treated individual or group of treated individuals as compared with an untreated individual or untreated group individuals.

123. The method of claim 120, wherein the elamipretide is administered subcutaneously once daily at 40 mg/dose.

124. The method of claim 120, wherein the subject has, or is suspected of having, geographic atrophy (GA) secondary to age-related macular degeneration (AMD).

125. The method of claim 120, wherein the subject has, or is suspected of having, non-exudative (dry) age-related macular degeneration.

126. The method of claim 120, wherein the subject has, or is suspected of having, intermediate age-related macular degeneration.

127. The method of claim 120, wherein the subject has, or is suspected of having, age-related macular degeneration with photoreceptor loss or non-exudative (dry) age-related macular degeneration with photoreceptor loss.

128. The method of claim 120, further comprising separately, sequentially, or simultaneously administering a second active agent.

129. The method of claim 128, wherein the second active agent comprises an AREDS or AREDS 2 vitamin formula.

130. The method of claim 128, wherein the second active agent is selected from the group consisting of: an antioxidant, a metal complexer, an anti-inflammatory drug, an antibiotic, and an antihistamine.

131. The method of claim 130, wherein the antioxidant is vitamin A, vitamin C, vitamin E, lycopene, selenium, α-lipoic acid, coenzyme Q, glutathione, or a carotenoid.

132. The method of claim 128, wherein the second active agent is mometasone furoate, tacrolimus, quercetin, or diphenhydramine.

133. The method of claim 128, wherein the second active agent is flavonoid, a coumarin, a phenol or a terpenoid.

134. The method of claim 133, wherein the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin.

135. The method of claim 133, wherein the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione).

136. The method of claim 133, wherein the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one).

137. The method of claim 133, wherein the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

138. The method of claim 128, wherein the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.
